(19) United States
(12) Patent Application Publication
(10) Pub. No.: US 2025/0205163 A1
(43) Pub. Date: Jun. 26, 2025

(54) ENGINEERED EXTRACELLULAR VESICLES FOR TARGETED DRUG DELIVERY TO MUSCLE

(71) Applicant: UNIVERSITY OF DELAWARE, Newark, DE (US)

(72) Inventors: Kyle Shuler, Elizabethtown, PA (US); Andrew Mitchell, Columbus, OH (US); Eric Muñoz, Phoenixville, PA (US); John Sperduto, Leonardo, NJ (US); Matthew Hudson, Media, PA (US)

(21) Appl. No.: 18/848,245

(22) PCT Filed: Mar. 23, 2023
(86) PCT No.: PCT/US2023/064840

§ 371 Date: Sep. 18, 2024

Related U.S. Application Data
(60) Provisional application No. 63/322,775, filed on Mar. 23, 2022.

(51) Int. Cl. A61K 9/50; A61K 45/06; C07K 14/47; C12N 5/071

(52) U.S. Cl. CPC A61K 9/5068; A61K 45/06; C07K 14/4703; C07K 14/4705; C12N 5/0686; C07K 2319/33

(57) ABSTRACT

The invention relates to myotropic extracellular vesicle compositions comprising a muscle-targeting membrane protein and optionally one or more therapeutic agents. The invention further relates to methods of using the myotropic extracellular vesicles comprising a muscle-targeting membrane protein for therapeutic applications for treating muscular disorders, conditions, and damage in a subject.

### STATEMENT OF PRIORITY

This application claims the benefit of U.S. Provisional Application Ser. No. 63/322,775, filed Mar. 23, 2022, the entire contents of which are incorporated by reference herein.

### GOVERNMENT SUPPORT

This invention was made with government support under Grant Numbers GM113125, NS102157, and GM103446 awarded by the National Institutes of Health. The government has certain rights in this invention.

### STATEMENT REGARDING ELECTRONIC FILING OF A SEQUENCE LISTING

A Sequence Listing in XML format, entitled 1653-2WO_ST26.xml, 2,684 bytes in size, generated on Mar. 21, 2023 and filed herewith, is hereby incorporated by reference in its entirety for its disclosures.

### FIELD OF THE INVENTION

This invention relates to myotropic extracellular vesicles and their use to selectively deliver one or more therapeutic agents to muscle cells/tissue. The invention further relates to methods of using extracellular vesicles for therapeutic applications for treating muscular diseases, conditions, and damage in a subject.

### BACKGROUND

Muscular pathologies comprise a wide range of diseases and disorders affecting the musculoskeletal system, including genetic conditions, such as muscular dystrophies, as well as secondary muscular pathologies, such as cancer cachexia. Overall, muscular pathologies represent an expansive category of diseases and disorders greatly impacting the lives of many. They range from inherited disorders arising from genetic mutations in genes encoding proteins that are critical to muscle function to acquired secondary pathologies as comorbidities of other conditions. Regardless of the cause, these are devastating conditions requiring advanced therapeutic interventions.

A number of treatment modalities exist for the treatment of muscular pathologies, including glucocorticoids and various gene and cell therapies. However, the only therapeutic currently approved by the United States Food and Drug Association (FDA) is glucocorticoid treatment while cell and gene therapies are still largely being studied in clinical trials and preclinical investigations. Moreover, although these treatment strategies have proven effective, they are non-specific to the musculature and thus suffer from off-target effects. The most promising therapeutics for these conditions are adeno-associated virus (AAV)-mediated gene therapies. They have shown promise in clinical trials; however, immunogenicity and lack of specificity of the AAV are major hurdles for clinical approval.

Extracellular vesicles (EVs), or cell-derived nanoparticles, have been proposed as alternative delivery vectors. By engineering targeting moieties on the surface of EVs, researchers have enhanced EV tropism to specific cell-types. However, no successful myotropic EV formulation currently exists. There is therefore a need for effective muscle-specific therapies with specificity and reduced immunogenicity.

### SUMMARY OF THE INVENTION

The present invention is based, in part, on the development of myotropic EVs. In an aspect, the invention relates to a composition comprising EVs isolated from cell culture medium of cultured cells modified to express a muscle targeting membrane protein, or fragment thereof, wherein the EVs are targeted to a muscle cell or tissue. The EVs may comprise one or more therapeutic agents.

An aspect of the invention relates to a method of making the compositions comprising EVs, comprising introducing a nucleic acid encoding the targeting membrane protein, or fragment thereof, into a cell; culturing the cell in cell culture medium to thereby express the target membrane protein; and isolating the composition comprising the EVs from the cell culture medium.

The invention further relates to a method of treating a muscle-related disorder, condition, or damage in a subject in need thereof, comprising administering a therapeutically effective amount of the EVs of the invention to the subject; thereby treating the disorder, condition, or damage.

A further aspect of the invention relates to a method of targeting one or more therapeutic agents to muscle cells, comprising contacting the muscle cells with EVs isolated from cell culture medium of cultured cells, the EVs modified to express a muscle targeting membrane protein, or fragment thereof, and one or more therapeutic agents.

These and other aspects of the invention are set forth in more detail in the description of the invention below.

### BRIEF DESCRIPTION OF THE DRAWINGS

FIGS. 1A-ID. FIG. 1A. Current therapeutic modalities are non-specific and result in adverse off-target effects in non-muscle organs. FIG. 1B. Myotropic-Extracellular Vesicles (MT-EV) drug delivery technology offers muscle-targeted delivery of therapeutics to minimize off-target effects. FIG. 1C. MTP-expressing stable cell line production through lentivirus transfection and cell selection via flow assisted cell sorting to generate a stable cell line. HEK293 cells were transfected with lentivirus constructs containing plasmids for selected MTPs containing an endogenous green fluorescent protein (GFP) tag for 24 hours. Following the transfection protocol, the cells were trypsinized and the cells that were successfully transfected were sorted via flow assisted cell sorting (FACS). The MTP-GFP expressing cells were then cultured as a stable cell line. FIG. 1D. Schematic overview of myotropic EV candidates.

FIGS. 2A-2F. Characterization of native myotropic EV candidates and producer cells. FIG. 2A. Transfection workflow used for generation of myotropic EV candidates. FIG. 2B. Confirmation of successful transfection, noting variance in transfection efficiency. FIG. 2C. Nanoparticle tracking analysis (NTA) data demonstrating concentration and size of particles in EV samples. FIG. 2D. Transmission electron microscopy images of each EV sample, large arrows, EVs, smaller arrows, serum-derived lipoproteins or protein aggregates. FIG. 2E. Flow cytometry workflow and data measuring incorporation of each myotropic protein candidate into EVs indicated relative to Non-Transfected-EVs (NT-EVs). NT: EVs from HEK293 cells that were not transfected; TR: EVs from cells that received only the transfection reagent; M-Cad: M-Cadherin; MYMK: MyoMaker; MYMX: MyoMixer; M&M: MyoMaker and MyoMixer. *p<0.05, **p<0.01, ns: not significant. N=3 independent experiments. FIG. 2F. Nanoparticle tracking analysis data demonstrating the modal size of the particulates isolated from the transduced HEK cell culture media or that of control cells falls within the range of EVs (30-200 nm).

FIGS. 3A-3B. Fluorescence data from FACS demonstrating the production efficiency of stable cell lines over-expressing the muscle-enriched membrane fusion protein, MyoMaker (MYMK), or a GFP control. FIG. 3A. Mean fluorescence intensity of the two stable cell lines. FIG. 3B. Percentage of total cells expressing the protein product of the transfected plasmid.

FIG. 4. Schematic of example MYMK and GFP cell culture/EV harvesting. Stable cells lines are cultured following FACS and scaled to larger cell culture vessels for EV harvesting or frozen down for future culture and/or further analysis.

FIG. 5. Mass spectrometry data outlining the top biological processes in which HEK0293 EV-derived proteins are involved, showing potentially therapeutic protein cargo involved various biological processes.

FIGS. 6A-6C. In vitro uptake assay to assess myotropic properties of each MT-EV candidate. FIG. 6A. Experimental design displaying each MT-EV formulation and control HEK293-EVs. Each formulation was incubated with fully differentiated C2C12 myotubes in vitro for 24 h prior to fluorescence imaging and analysis. FIG. 6B. Representative 10× fluorescence images depicting the labeled protein cargo (gray) delivered into the myotubes, identified using a nuclear stain (dark gray). FIG. 6C. Quantification of the fluorescence intensity of the labeled protein delivered into the myotubes by each EV candidate relative to NT-EVs. NT: EVs from cells that were not transfected; TR:EVs from HEK293 cells that received only the transfection reagent; M-Cad: M-Cadherin; MYMK: MyoMaker; MYMX: MyoMixer; M&M: MyoMaker and MyoMixer. ***p<0.001. N=3 independent experiments.

FIGS. 7A-7B. Comparison of cationic reagent and lentiviral transfection workflows for the generation of a stable MYMK-expressing cell line. FIG. 7A. Cationic lipid transfection workflow and data demonstrating an 89% decline in fluorescent signal over 10 passages following cationic lipid-based transfection. FIG. 7B. Lentiviral transfection workflow and data demonstrating a 19% decline in fluorescent signal over 10 passages following lentiviral transfection and selection using fluorescence-assisted cell sorting. Data presented as mean fluorescence intensity. MFI: mean fluorescence intensity. ***p<0.001. N=3 independent experiments.

FIGS. 8A-8E. Characterization data for EVs produced by stable MYMK-expressing cell line and HEK293 control cell line and producer cells. FIG. 8A. NTA histogram of MYMK and HEK EVs depicting concentration and size distribution. FIG. 8B. Table depicting concentration, size, and protein content of HEK and MYMK-EVs. FIG. 8C. Fluorescence intensity of MYMK-producer cells over multiple passages (P19-23) during EV production as compared to HEK293 control cells. FIG. 8D. Representative TEM images of HEK and MYMK-EV samples. FIG. 8E. Western blot analysis identifying the presence of EV and non-EV-enriched proteins in the EV samples as well as ultracentrifuge supernatant (SN) and producer cell lysate (CL). *p<0.05. ns: not significant. N=3 independent experiments.

FIGS. 9A-9C. In vivo biodistribution of DiD-labeled MYMK and HEK-EVs. FIG. 9A. Experimental groups for injections into tail vein of C57 and mdx mice. FIG. 9B. Quantitative analysis of relative fluorescence intensity of homogenized tissue 24 h after injection, normalized by tissue weight (g) and non-injected tissue signal subtracted. FIG. 9C. Representative images of organs from C57 and mdx mice 24 h following injections taken on in vivo imaging system. N=18 total mice, 3 mice per group. NI=No Injection. HEK=HEK293-EV Injection. MYMK=MYMK-EV injection. *p<0.05, **p<0.01, ****p<0.0001.

FIGS. 10A-10E. In vivo biodistribution of HEK and MYMK-EVs labeled with an amine-reactive probe. FIG. 10A. Particle concentration, mean and modal size of EVs labeled with CellTracker-Red or DiD (N=2 independent experiments). FIG. 10B. Fluorescence signal of EVs labeled with amine-reactive probe. FIG. 10C. Uptake of amine-labeled EVs by HEK293 cells in vitro (10×). FIG. 10D. Quantitative analysis of relative fluorescence intensity of homogenized tissue 24 h after injection, normalized by tissue weight (g) and noninjected tissue signal subtracted. FIG. 10E. Representative 40× fluorescent images of tissues of interest (gastrocnemius and heart) and off-target tissues known to take up the majority of intravenously administered EVs (liver and spleen) labeled with phalloidin 488 conjugate (gray) and DAPI (dark gray). For in vivo experiments, N=18 total mice, 3 mice per group. For non-animal experiments, N=3 independent experiments unless otherwise indicated.

FIG. 11. Capillary western blot analyzing HEK293 cell lysate (CL), UC supernatant (SN), HEK293 EVs, PTGFRN EVs, MP1 EVs and MP2 EVs confirming signal for ALIX (100 kDa) in the CL, SN, PTGFRN and MP1 samples but not the HEK and MP2 due to low particle/protein concentrations in those samples.

FIG. 12. Fluorescence intensity of Cell Tracker Red-labeled chimeric myotropic EV candidates at 1:10 dilution. Data were used to calculate doses of EVs for in vitro uptake experiments.

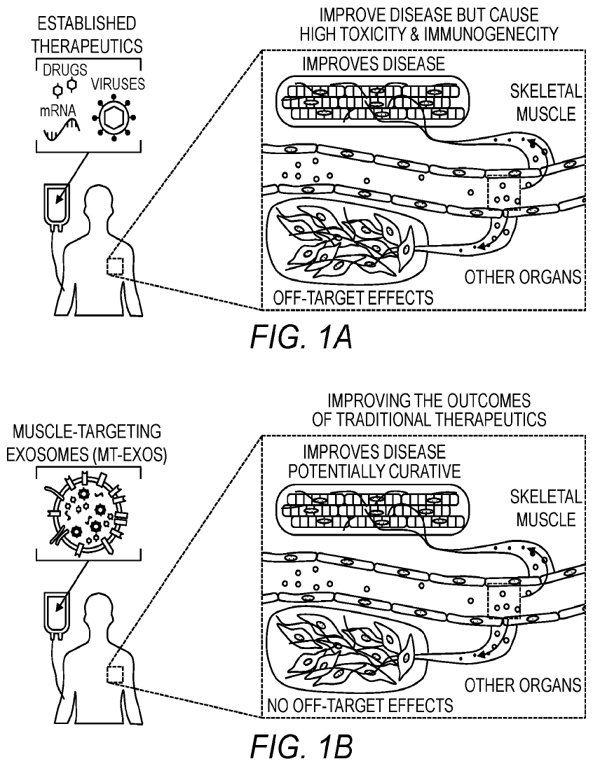

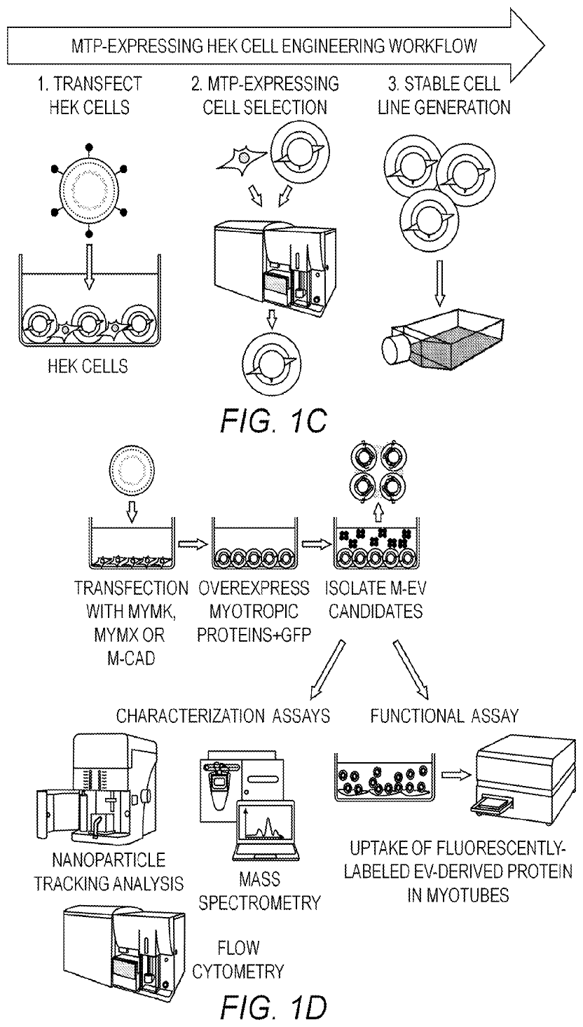

### DETAILED DESCRIPTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art. In addition, any references cited herein are incorporated by reference in their entireties.

Unless otherwise defined, all technical and scientific terms used herein have the same meaning as commonly understood by one of skill in the art to which this invention belongs. The terminology used in the description of the invention herein is for the purpose of describing particular embodiments only and is not intended to be limiting of the invention. All publications, patent applications, patents, patent publications and other references cited herein are incorporated by reference in their entireties for the teachings relevant to the sentence and/or paragraph in which the reference is presented.

Amino acids are represented herein in the manner recommended by the IUPAC-IUB Biochemical Nomenclature Commission, or (for amino acids) by either the one-letter code, or the three-letter code, both in accordance with 37 C.F.R. § 1.822 and established usage.

Except as otherwise indicated, standard methods known to those skilled in the art may be used for cloning genes, amplifying and detecting nucleic acids, and the like. Such techniques are known to those skilled in the art. See, e.g., Sambrook et al., Molecular Cloning: A Laboratory Manual 4th Ed. (Cold Spring Harbor, NY, 2012); Ausubel et al. Current Protocols in Molecular Biology (Green Publishing Associates, Inc. and John Wiley & Sons, Inc., New York).

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate, if the specification states that a complex comprises components A, B and C, it is specifically intended that any of A, B or C, or a combination thereof, can be omitted and disclaimed singularly or in any combination.

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of ±10%, ±5%, 1%, ±0.5%, or even ±0.1% of the specified amount.

As used herein, the transitional phrase “consisting essentially of” (and grammatical variants) is to be interpreted as encompassing the recited materials or steps and those that do not materially affect the basic and novel characteristic(s) of the claimed invention. Thus, the term “consisting essentially of” as used herein should not be interpreted as equivalent to “comprising.”

The term “consists essentially of” (and grammatical variants), as applied to a polypeptide or polynucleotide sequence of this invention, means a polypeptide or polynucleotide that consists of both the recited sequence (e.g., SEQ ID NO) and a total of ten or less (e.g., 1, 2, 3, 4, 5, 6, 7, 8, 9, or 10) additional amino acids on the N-terminal and/or C-terminal ends of the recited sequence or additional nucleotides on the 5′ and/or 3′ ends of the recited sequence such that the function of the polypeptide or polynucleotide is not materially altered. The total of ten or less additional amino acids or nucleotides includes the total number of additional amino acids or nucleotides on both ends added together. The term “materially altered,” as applied to polypeptides of the invention, refers to an increase or decrease in biological activities/properties of at least about 50% or more as compared to the activity of a polypeptide consisting of the recited sequence.

The term “sequence identity,” as used herein, has its standard meaning in the art. As is known in the art, a number of different programs can be used to identify whether a polynucleotide or polypeptide has sequence identity or similarity to a known sequence. Sequence identity or similarity may be determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12:387 (1984), preferably using the default settings, or by inspection

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351 (1987); the method is similar to that described by Higgins & Sharp, CABIOS 5:151 (1989).

Another example of a useful algorithm is the BLAST algorithm, described in Altschul et al., J. Mol. Biol. 215:403 (1990) and Karlin et al., Proc. Natl. Acad. Sci. USA 90:5873 (1993). A particularly useful BLAST program is the WU-BLAST-2 program which was obtained from Altschul et al., Meth. Enzymol., 266:460 (1996); blast.wustl/edu/blast/README.html. WU-BLAST-2 uses several search parameters, which are preferably set to their default values. The parameters are dynamic values and are established by the program itself depending upon the composition of the particular sequence of interest and the composition of the particular database against which the sequence of interest is being searched; however, the values may be adjusted to increase sensitivity.

An additional useful algorithm is gapped BLAST as reported by Altschul et al., Nucleic Acids Res. 25:3389 (1997).

A percentage amino acid sequence identity value is determined by the number of matching identical residues divided by the total number of residues of the “longer” sequence in the aligned region. The “longer” sequence is the one having the most actual residues in the aligned region (gaps introduced by WU-BLAST-2 to maximize the alignment score are ignored).

In a similar manner, percent nucleic acid sequence identity is defined as the percentage of nucleotide residues in the candidate sequence that are identical with the nucleotides in the polynucleotide specifically disclosed herein.

The alignment may include the introduction of gaps in the sequences to be aligned. In addition, for sequences that contain either more or fewer nucleotides than the polynucleotides specifically disclosed herein, it is understood that in one embodiment, the percentage of sequence identity will be determined based on the number of identical nucleotides in relation to the total number of nucleotides. Thus, for example, sequence identity of sequences shorter than a sequence specifically disclosed herein, will be determined using the number of nucleotides in the shorter sequence, in one embodiment. In percent identity calculations, relative weight is not assigned to various manifestations of sequence variation, such as insertions, deletions, substitutions, etc.

In one embodiment, only identities are scored positively (+1) and all forms of sequence variation including gaps are assigned a value of “0,” which obviates the need for a weighted scale or parameters as described below for sequence similarity calculations. Percent sequence identity for example, 80%, 85%, 90%, or 95%, can be calculated, for example, by dividing the number of matching identical residues by the total number of residues of the “shorter” sequence in the aligned region and multiplying by 100. The “longer” sequence is the one having the most actual residues in the aligned region.

As used herein, an “isolated” nucleic acid or nucleotide sequence (e.g., an “isolated DNA” or an “isolated RNA”) means a nucleic acid or nucleotide sequence separated or substantially free from at least some of the other components of the naturally occurring organism or virus, for example, the cell or viral structural components or other polypeptides or nucleic acids commonly found associated with the nucleic acid or nucleotide sequence.

Likewise, an “isolated” polypeptide means a polypeptide that is separated or substantially free from at least some of the other components of the naturally occurring organism or virus, for example, the cell or viral structural components or other polypeptides or nucleic acids commonly found associated with the polypeptide.

As used herein, by “isolate” or “purify” (or grammatical equivalents) a virus vector, it is meant that the virus vector is at least partially separated from at least some of the other components in the starting material.

The term “endogenous” refers to a component naturally found in an environment, i.e., a gene, nucleic acid, miRNA, protein, cell, or other natural component expressed in the subject, as distinguished from an introduced component, i.e., an “exogenous” component.

As used herein, the term “heterologous” refers to a nucleotide/polypeptide that originates from a foreign species, or, if from the same species, is substantially modified from its native form in composition and/or genomic locus by deliberate human intervention.

As used herein, the term “nucleic acid” refers to a single or double-stranded polymer of deoxyribonucleotide or ribonucleotide bases read from the 5′ to the 3′ end. The “nucleic acid” may also optionally contain non-naturally occurring or modified nucleotide bases. The term “nucleotide sequence” or “nucleic acid sequence” refers to both the sense and antisense strands of a nucleic acid, either as individual single strands or in the duplex. The term “ribonucleic acid” (RNA) is inclusive of RNAi (inhibitory RNA), dsRNA (double stranded RNA), siRNA (small interfering RNA), shRNA (short/small hairpin RNA), mRNA (messenger RNA), miRNA (micro-RNA), tRNA (transfer RNA, whether charged or discharged with a corresponding acylated amino acid), long non-coding RNA (lncRNA), ribosomal RNA (rRNA), small nuclear RNA (snRNA), small nucleolar RNA (snoRNA) and cRNA (complementary RNA), and the term “deoxyribonucleic acid” (DNA) is inclusive of cDNA and genomic DNA and DNA-RNA hybrids.

The terms “nucleic acid segment,” “nucleotide sequence,” or more generally “segment” will be understood by those in the art as functional terms that include genomic sequences, ribosomal RNA sequences, transfer RNA sequences, messenger RNA sequences, small regulatory RNAs, operon sequences and smaller engineered nucleotide sequences that express or may be adapted to express, proteins, polypeptides or peptides. Nucleic acids of the present disclosure may also be synthesized, either completely or in part, by methods known in the art. Thus, all or a portion of the nucleic acids of the present disclosure may be synthesized using codons preferred by a selected host. Such species-preferred codons may be determined, for example, from the codons used most frequently in the proteins expressed in a particular host species. Other modifications of the nucleotide sequences may result in mutants having slightly altered activity.

As used herein with respect to nucleic acids, the term “fragment” refers to a nucleic acid that is reduced in length relative to a reference nucleic acid and that comprises, consists essentially of and/or consists of a nucleotide sequence of contiguous nucleotides identical or almost identical (e.g., 90%, 91%, 92%, 93%, 94%, 95%, 96%, 97%, 98%, 99% identical) to a corresponding portion of the reference nucleic acid. Such a nucleic acid fragment may be, where appropriate, included in a larger polynucleotide of which it is a constituent. In some embodiments, the nucleic acid fragment comprises, consists essentially of or consists of at least about 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, or more consecutive nucleotides. In some embodiments, the nucleic acid fragment comprises, consists essentially of or consists of less than about 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450 or 500 consecutive nucleotides.

As used herein with respect to polypeptides, the term “fragment” refers to a polypeptide that is reduced in length relative to a reference polypeptide and that comprises, consists essentially of and/or consists of an amino acid sequence of contiguous amino acids identical or almost identical (e.g., 90%, 91%, 92%, 93%, 94%, 95%, 96%, 97%, 98%, 99% identical) to a corresponding portion of the reference polypeptide. Such a polypeptide fragment may be, where appropriate, included in a larger polypeptide of which it is a constituent. In some embodiments, the polypeptide fragment comprises, consists essentially of or consists of at least about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, or more consecutive amino acids. In some embodiments, the polypeptide fragment comprises, consists essentially of or consists of less than about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450 or 500 consecutive amino acids.

As used herein with respect to nucleic acids, the term “functional fragment” or “active fragment” refers to nucleic acid that encodes a functional fragment of a polypeptide.

As used herein with respect to polypeptides, the term “functional fragment” or “active fragment” refers to polypeptide fragment that retains at least about 20%, 25%, 30%, 35%, 40%, 45%, 50%, 55%, 60%, 65%, 70%, 75%, 80%, 85%, 90%, 95%, 96%, 97%, 98%, 99%, 99.5% or more of at least one biological activity of the full-length polypeptide. In some embodiments, the functional fragment actually has a higher level of at least one biological activity of the full-length polypeptide.

As used herein, the term “modified,” as applied to a polynucleotide or polypeptide sequence, refers to a sequence that differs from a wild-type sequence due to one or more deletions, additions, substitutions, or any combination thereof.

The terms “enhance” and “increase” refer to an increase in the specified parameter of at least about 1.25-fold, 1.5-fold, 2-fold, 3-fold, 4-fold, 5-fold, 6-fold, 8-fold, 10-fold, twelve-fold, or even fifteen-fold.

The terms “inhibit” and “reduce” or grammatical variations thereof as used herein refer to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectible entity or activity (at most, an insignificant amount, e.g., less than about 10% or even 5%).

As used herein, “expression” refers to the process by which a polynucleotide is transcribed from a DNA template (such as into an mRNA or other RNA transcript) and/or the process by which a transcribed mRNA is subsequently translated into peptides, polypeptides, or proteins. Transcripts may be referred to as “transcription products” and encoded polypeptides may be referred to as “translation products.” Transcripts and encoded polypeptides may be collectively referred to as “gene products.” If the polynucleotide is derived from genomic DNA, expression may include splicing of the mRNA in a eukaryotic cell. The expression product itself, e.g., the resulting nucleic acid or protein, may also be said to be “expressed.” An expression product can be characterized as intracellular, extracellular or secreted. The term “intracellular” means something that is inside a cell. The term “extracellular” means something that is outside a cell. A substance is “secreted” by a cell if it appears in significant measure outside the cell, from somewhere on or inside the cell.

As used herein, the term “synthetic gene” refers to a nucleic acid sequence generated non-naturally by deliberate human design, the synthetic gene comprising, among other components, a coding region for a protein or nucleic acid of interest, and regulatory regions for expression of the coding region. Structural and functional components of the synthetic gene may be incorporated from differing and/or a plurality of source material. The synthetic gene may be delivered exogenously to a subject, wherein it would be exogenous in comparison to a corresponding endogenous gene. When expressed in a cell, the synthetic gene product may be referred to as a synthetic product (e.g., “synthetic RNA” or “synthetic polypeptide”). Under certain conditions, the synthetic gene may also be interchangeably referred to as a “transgene.”

As used herein, the terms “transgenic” and/or “transgene” refer to a nucleic acid sequence containing a functional coding region for a gene that comprises one or more exogenous nucleic acids. The exogenous nucleic acid can be stably integrated within the genome such that the polynucleotide is passed on in successive cell divisions. The exogenous nucleic acid can be integrated into the genome alone or as part of a recombinant expression cassette. “Transgenic” may be used to designate any substrate the genotype of which has been altered by the presence of an exogenous nucleic acid.

The terms “polypeptide,” “peptide” and “protein” may be used interchangeably to refer to polymers of amino acids of any length. The terms “nucleic acid,” “nucleic acid sequence,” and “polynucleotide” may be used interchangeably to refer to polymers of nucleotides of any length. As used herein, the terms “nucleotide sequence,” “polynucleotide,” “nucleic acid sequence,” “nucleic acid molecule” and “nucleic acid fragment” refer to a polymer of RNA, DNA, or RNA and DNA that is single- or double-stranded, optionally containing synthetic, non-natural and/or altered nucleotide bases.

As used herein, the terms “gene of interest,” “nucleic acid of interest” and/or “protein of interest” refer to that gene/nucleic acid/protein desired under specific contextual conditions.

The term “regulatory element” refers to a genetic element which controls some aspect of the expression of nucleic acid sequences. For example, a promoter is a regulatory element that facilitates the initiation of transcription of an operably linked coding region. Other regulatory elements are splicing signals, polyadenylation signals, termination signals, etc. The region in a nucleic acid sequence or polynucleotide in which one or more regulatory elements are found is referred to as a “regulatory region.”

The term “coding region” as used herein, refers to the portion of a polynucleotide, e.g., a gene, that encodes a polypeptide.

As used herein with respect to nucleic acids, the term “operably linked” refers to a functional linkage between two or more nucleic acids. For example, a promoter sequence may be described as being “operably linked” to a heterologous nucleic acid sequence because the promoter sequences initiates and/or mediates transcription of the heterologous nucleic acid sequence. In some embodiments, the operably linked nucleic acid sequences are contiguous and/or are in the same reading frame.

A “vector” refers to a compound used as a vehicle to carry foreign genetic material into another cell, where it can be replicated and/or expressed. A cloning vector containing foreign nucleic acid is termed a recombinant vector. Examples of nucleic acid vectors are plasmids, viral vectors, cosmids, expression cassettes, and artificial chromosomes. Recombinant vectors typically contain an origin of replication, a multicloning site, and a selectable marker. The nucleic acid sequence typically consists of an insert (recombinant nucleic acid or transgene) and a larger sequence that serves as the “backbone” of the vector. The purpose of a vector which transfers genetic information to another cell is typically to isolate, multiply, or express the insert in the target cell. Expression vectors (expression constructs or expression cassettes) are for the expression of the exogenous gene in the target cell, and generally have a promoter sequence that drives expression of the exogenous gene. Insertion of a vector into the target cell is referred to transformation or transfection for bacterial and eukaryotic cells, although insertion of a viral vector is often called transduction. The term “vector” may also be used in general to describe items to that serve to carry foreign genetic material into another cell, such as, but not limited to, a transformed cell or a nanoparticle.

As used herein, the term “adeno-associated virus” (AAV) includes but is not limited to, AAV serotype 1 (AAV1), AAV2, AAV3 (including types 3A and 3B), AAV4, AAV5, AAV6, AAV7, AAV8, AAV9, AAV10, AAV11, avian AAV, bovine AAV, canine AAV, equine AAV, ovine AAV, and any other AAV now known or later discovered. See, e.g., BERNARD N. FIELDS et al., VIROLOGY, volume 2, chapter 69 (4th ed., Lippincott-Raven Publishers). Recently, a number of putative new AAV serotypes and clades have been identified (see, e.g., Gao et al., (2004) J. Virology 78:6381-6388; Moris et al., (2004) Virology 33-:375-383; and Table 1). An AAV can be selected for tissue-specific delivery. AAV9 variants, for example AAV-PHP.B can be used for where desired to cross the blood brain barrier. AAV variants with reduced immunogenicity may also be utilized, and may comprise chimeric AAV, for example, AAV-DJ. Design strategies for AAV vectors may also be employed for the delivery of the [modified protein] according to the present invention. See, e.g., Lee, et al. (2018) Adeno-associated virus (AAV) vectors: rational design strategies for capsid engineering. Curr. Opin. Biomed. Eng., 7, 58-63; see also Parambi et al., 2021 Oct. 15, Mol Neurobiol. 2022; 59(1): 191-233, doi:10.1007/s12035-021-02555-y, incorporated herein by reference in its entirety, and specifically Table 1 for teachings of viral vectors.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

As used herein, the term “host cell” refers to a cell that is engineered to express the modified polypeptide or functional fragment thereof (e.g., a modified full length [protein] or a fragment thereof). “Host cell” refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

Host cells may be derived from prokaryotes or eukaryotes, depending upon whether the desired result is replication of the vector or expression of part or all of the vector-encoded nucleic acid sequences. Prokaryotes include gram negative or positive cells. Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5α, JM109, and KC8, as well as a number of commercially available bacterial hosts such as SURE® Competent Cells and SOLOPACK™ Gold Cells (STRATAGENE®, La Jolla). Alternatively, bacterial cells such as E. coli LE392 could be used as host cells for phage viruses.

By “pharmaceutically acceptable” it is meant a material that is not toxic or otherwise undesirable, i.e., the material may be administered to a subject without causing any undesirable biological effects.

“Pharmaceutically acceptable carrier” (sometimes referred to as a “carrier”) refers to a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

By the terms “treat,” “treating,” and “treatment of” (or grammatically equivalent terms) it is meant that the severity of the subject's condition is reduced or at least partially improved or ameliorated and/or that some alleviation, mitigation or decrease in at least one clinical symptom is achieved and/or there is a delay in the progression of the condition. Treatment may include, but is not limited to, administering an agent or composition (e.g., a pharmaceutical composition) to a subject. Treatment is typically undertaken in an effort to alter the course of a disease (which term is used to indicate any disease, disorder, syndrome or undesirable condition warranting or potentially warranting therapy) in a manner beneficial to the subject. The effect of treatment may include reversing, alleviating, reducing severity of, delaying the onset of, curing, inhibiting the progression of, and/or reducing the likelihood of occurrence or recurrence of the disease or one or more symptoms or manifestations of the disease. A therapeutic agent, e.g., extracellular vesicle, may be administered to a subject who has a disease or is at increased risk of developing a disease relative to a member of the general population. In some embodiments a therapeutic agent may be administered to a subject who has had a disease but no longer shows evidence of the disease. The agent may be administered e.g., to reduce the likelihood of recurrence of evident disease. A therapeutic agent may be administered prophylactically, i.e., before development of any symptom or manifestation of a disease. “Prophylactic treatment” refers to providing medical and/or surgical management to a subject who has not developed a disease or does not show evidence of a disease in order, e.g., to reduce the likelihood that the disease will occur, delay the onset of the disease, or to reduce the severity of the disease should it occur. The subject may have been identified as being at risk of developing the disease (e.g., at increased risk relative to the general population or as having a risk factor that increases the likelihood of developing the disease.

As used herein, the terms “prevent,” “prevents,” and “prevention” (and grammatical equivalents thereof) refer to a delay in the onset of a disease or disorder or the lessening of symptoms upon onset of the disease or disorder. The terms are not meant to imply complete abolition of disease and encompass any type of prophylactic treatment that reduces the incidence of the condition or delays the onset and/or progression of the condition.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

The terms “administering” and “administration” of a synthetic gene, expression cassette, vector, plasmid, viral vector, transformed cell, nanoparticle (including all extracellular vesicles), or pharmaceutical composition to a subject include any route of introducing or delivering to a subject a compound to perform its intended function. Administration can be carried out by any suitable route, including orally, intranasally, parenterally (intravenously, intramuscularly, intraperitoneally, intracisternally, intrathecally, intraventricularly, or subcutaneously), or topically. Administration includes self-administration and administration by another. “Concurrent administration,” “administration in combination,” “simultaneous administration,” or “administered simultaneously” as used herein, means that the compounds are administered at the same point in time, overlapping in time, or one following the other. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g., greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

The term “nuclear localization signal,” or “nuclear localization sequence,” (“NLS”) as used herein refers to an amino acid sequence that tags a protein for import into the cell nucleus by cell transport.

Those skilled in the art will appreciate that a variety of promoters may be used depending on the level and specific expression desired. The promoter may be constitutive or regulatable, depending on the pattern of expression desired. The promoter may be native or foreign and can be a natural or a synthetic sequence. By foreign, it is intended that the transcriptional initiation region is not found in the wild-type host into which the transcriptional initiation region is introduced.

The promoter can be native to the target cell or subject to be treated and/or can be native to the heterologous nucleotide sequence. The promoter is generally chosen so that it will function in the target cell(s) of interest. The promoter can optionally be a mammalian promoter. The promoter may further be constitutive or regulatable (e.g., inducible).

Promoters for nucleic acid delivery can be tissue preferred and/or -specific promoters. In some embodiments, the promoter is brain-specific or brain-preferred, spinal cord-specific or spinal cord-preferred, or muscle-specific or muscle-preferred.

The term “modulate,” “modulates,” or “modulation” refers to enhancement (e.g., an increase) or inhibition (e.g., a decrease) in the specified level or activity.

The term “contact” or grammatical variations thereof as used with respect to an EV and a cell, refers to bringing the EV and the cell in sufficiently close proximity to each other for one to exert a biological effect on the other. This may include forms of administration, as described further herein. In some embodiments, the term contact results in uptake of the EV into the cell.

A “subject” may be any vertebrate organism in various embodiments. A subject may be individual to whom an agent is administered, e.g., for experimental, diagnostic, and/or therapeutic purposes or from whom a sample is obtained or on whom a procedure is performed. In some embodiments a subject is a mammal, e.g., a human, non-human primate, lagomorph (e.g., rabbit), or rodent (e.g., mouse, rat). In some embodiments a human subject is a neonate, child, adult or geriatric subject. In some embodiments a human subject is at least 50, 60, 70, 80, or 90 years old.

As used herein, the term “extracellular vesicles” refers to lipid-bound particles that are released from cells. The term encompasses, without limitation, exosomes, ectosomes, microvesicles, microparticles, nanoparticles, large EVs, and autophagosomes, and range in diameter from 20-30 nm to 10 microns or more. Extracellular vesicles carry a cargo of proteins, nucleic acids, lipids, metabolites, and/or organelles from the parent cell.

As used herein, the term “muscle-related disorder, condition, or damage” refers to a disorder, condition, or damage that occurs in muscle cells or tissue and may include skeletal muscle, cardiac muscle, and/or smooth muscle. The disorder, condition, or damage may be due to an issue (e.g., mutation or trauma) that originates in muscle cells or tissue or to an issue that originates outside of muscle cells or tissue but results in a disorder, condition, or damage in muscle.

### Compositions

Compositions provided herein comprise EVs isolated from cell culture medium (e.g., supernatant, broth, harvest fluid) of cultured cells modified to express a muscle targeting membrane protein, or fragment thereof, that will target the EVs to muscle cells or tissue. In an embodiment, the muscle tissue is skeletal tissue or cardiac tissue.

EVs are a heterogeneous class of cell-derived nanoparticles varying in size (30-1000 nm) and mechanism of biogenesis. Due to their biocompatibility and engineerability, EVs are potential vectors for biotherapeutics. More specifically, EVs can be engineered to display various targeting moieties, altering the tropism of the EVs for targeted delivery of cargo to specific cell/tissue types. EVs of the present invention are designed to target muscle cells and tissue.

In some embodiments, the muscle targeting membrane protein of the extracellular vesicle can be any protein listed in Table 1. In an embodiment, the targeting membrane protein is a native protein involved in myogenic fusion and adherence in skeletal muscle development. In an aspect, the protein is displayed on the surface of the EV. In an aspect, the protein can be displayed on a protein on the surface of the EV. In an embodiment, the muscle targeting membrane protein, or fragment thereof, is MyoMaker (MYMK), MyoMixer (MYMX), or M-Cadherin (M-CAD), or a combination thereof. In an embodiment, the muscle targeting membrane protein, or fragment thereof, is prostaglandin F2 receptor inhibitor (PTGFRN). In an embodiment, the PTGFRN can be fused to a synthetic myotropic peptide. The synthetic myotropic peptide can be ASSLNIA (MP1) (SEQ ID NO:1) or RRQPPRSISSHP (MP2) (SEQ ID NO:2). The synthetic myotropic peptide can be displayed on, e.g., fused to, a protein on the surface of the EV, including by not limited to, PTGFRN and may be fused, for example, to the N-terminal or C-terminal.

The composition can comprise EVs comprising one or more therapeutic agents. In an embodiment, the culture cells from which the EVs are derived have been modified to contain a higher level of the one or more therapeutic agents than unmodified cells. As described further herein, the one or more therapeutic agents may be a protein, RNA (mRNA, siRNA, microRNA, lncRNA), DNA, plasmid, viral vector (e.g., AAV), exon skipping compound, or any combination thereof. In an embodiment, the one or more therapeutic agents is selected from the agents listed in Table 2. In some embodiments, the one or more therapeutic agents is endogenously expressed in the cultured cells. In other embodiments, the one or more therapeutic agents or a nucleic acid encoding the one or more therapeutic agents can be introduced into the cultured cells from which the EVs are isolated. The compositions can be designed to target cardiac muscle or skeletal muscle tissue.

The cultured cells may be any cells that comprise a therapeutic agent that is present in EVs derived from the cells. In some embodiments, the cells are HEK293 cells. In an aspect, cells can be primary muscle cells or muscle cell lines. In some embodiments, the cells are muscle satellite cells, primary muscle cells, HEK293 cells, HEK293T cells, CAP cells, mesenchymal stem cells, immune cells, or any combination thereof.

### Targeting Agent

In some embodiments, the cells have been modified to contain a targeting agent that will target the extracellular vesicles to a target tissue. In some embodiments, the targeting agent is a targeting membrane protein, or fragment thereof. In some embodiments, the target tissue is muscle. The targeting agent may be, without limitation, one of the proteins listed in Table 1 or a functional fragment thereof.

TABLE 1
Muscle-targeting membrane proteins

| | Gene | Exemplary Sequence | Evidence |
|---|---|---|---|
| Protein | Symbol | Reference No. | level |
| MyoMaker | MYMK | NM_001080483.3 | Protein |
| MyoMixer | MYMX | XM_024446300 | Protein |
| M-Cadherin | CDH15 | NG_012055, | Protein |
| | | NM_004933.3 | |
| Sarcoglycan alpha | SGCA | NG_008889, | Protein |
| | | NM_000023.4 | |
| Sarcoglycan beta | SGCB | NG_008891, | Protein |
| | | NM_000232.5 | |
| Sarcoglycan gamma | SGCG | NG_008759.1, | Protein |
| | | NM_000231.3 | |
| Sarcospan | SSPN | NG_012011, | Protein |
| | | NM_005086.5 | |
| Dystroglycan alpha | DAGl | NM_004393; | Protein |
| | | NM_001165928.4 | |
| Dystroglycan beta | DAGl | NM_004393; | Protein |
| | | NM_001165928.4 | |
| Myoferlin | MYOF | NM_013451.4 | Protein |
| Dysferlin | DYSF | NM_001130455.2 | Protein |
| Cardiomyopathy- | CMYA5 | NM_153610.5 | Protein |
| associated protein 5 | | | |
| EH domain- | EHD2 | NM_014601.4 | Protein |
| containing protein 2 | | | |
| Cadherin 4 | CDH4 | NM_001252338.2 | Protein |

### Therapeutic Agents

One aspect of the invention relates to the use of EVs as delivery vehicles for therapeutic agents. In some embodiments, the EVs may naturally contain the therapeutic agents. In other embodiments, the cells from which the EVs are derived may be modified to contain the therapeutic agents or higher levels of the therapeutic agent, which then end up in the EVs. The use of EVs to deliver therapeutic agents such as viral vectors that may raise an immune response may prevent or diminish the immune response against the vector. Additionally, EVs can cross most membranes, including the blood brain barrier, so they may be used to deliver therapeutic agents to the brain as well as other tissues and organs.

The therapeutic agent may be any therapeutic agent know or later discovered to be effective for treatment of a disorder, condition, or damage, e.g., a muscle-related disorder, condition, or damage. In some embodiments, the therapeutic agent may be a protein, RNA (e.g., mRNA, siRNA, microRNA, lncRNA), DNA, expression vector (e.g., plasmid or viral vector (e.g., AAV), organelle, CRISPR complex, exon skipping compound (e.g., a morpholino exon skipping compound), or any combination thereof. In some embodiments, the therapeutic agent is a naturally occurring protein (e.g., dystrophin) or a nucleic acid encoding the naturally occurring protein. In some embodiments, the therapeutic agent is a non-naturally occurring protein (e.g., a fragment or modified version such as a minidystrophin or microdystrophin) or a nucleic acid encoding the non-naturally occurring protein.

A therapeutic agent can include one or more exemplary proteins associated with one of the muscular disorders listed in Table 2. The muscle-related disorder, condition, or damage may be any disorder, condition, or damage known to be or later discovered to be correlated with a biomarker found in muscle-derived EVs. The biomarker may be one that is directly related to the cause of the disorder, condition, or damage (e.g., a mutated gene) or one that indirectly related to the disorder, condition, or damage (e.g., as a consequence of the mutated gene). Non-limiting examples of muscle-related disorders, conditions, or damage and the proteins and nucleic acids associated with them are listed in Table 2.

TABLE 2

| | | | |
|---|---|---|---|
| | | | Exemplary |
| Disease | Genetic Mutation | Protein | Accession No. |

Congenital Muscular Dystrophies

| | | | |
|---|---|---|---|
| CMD with | Titin | Titin | CAA62188 |
| cardiomyopathy | | | |
| CMD with desmin | SEPN1 or Selenon | selenoprotein N | NP_996809 |
| inclusions (abnormal | | | |
| accumulations of the | | | |
| muscle protein desmin in | | | |
| some muscle fibers) | | | |
| CMD with integrin alpha | integrin-alpha 7 | integrin-alpha 7 | AAC18968 |
| 7 mutations | | | |
| CMD with joint | integrin alpha 9 | integrin alpha 9 | EAW64501 |
| hyperlaxity | | | |
| CMD with familial | plectin protein | plectin protein | CAA91196 |
| junctional epidermolysis | | | |
| bullosa | | | |
| CMD with muscle | fukutin-related protein | fukutin-related protein | AAH02612 |
| hypertrophy | (FKRP) | (FKRP) | |
| MDC1C | fukutin-related protein | fukutin-related protein | AAH02612 |
| | (FKRP) | (FKRP) | |
| MDC1D | LARGE gene | LARGE xylosyl- and | CAG30396 |
| | | glucuronyltransferase 1 | |
| CMD with myasthenic | DOK7 gene | docking protein 7 | NP_001288000 |
| syndrome | | | XP_005248013 |
| CMD with (early) spinal | SEPN1 or Selenon | selenoprotein N | NP_996809 |
| rigidity | | | |
| CMD with spinal | lamin A or C proteins | lamin A or C proteins | NP_001269554, |
| rigidity and lamin A/C | | | NP_733821 |
| abnormality | | | |
| CMD with spinal | SBP2 gene | SECIS binding protein 2 | AAH01189 |
| rigidity and | | | |
| selenoprotein deficiency | | | |
| CMD with structural | choline kinase beta gene | choline kinase beta | AAI01489 |
| abnormalities of | | | |
| mitochondria (energy- | | | |
| producing subunits of | | | |
| cells) | | | |
| MDDGA4 | fukutin gene | fukutin | BAA94082 |
| MDC1A | laminin alpha 2 gene | laminin alpha 2 | EAW48082 |
| Santavuori muscle-eye- | POMGnT1 gene | protein O-linked mannose N- | NP_001230695 |
| brain disease | | acetylglucosaminyltransferase 1 | |
| | | (beta 1,2-) | |
| Ullrich CMD | COLGA1, COL6A2 or | collagen type VI alpha 1 chain, 2 | AAH52575, |
| | COL6A3 genes | chain, and 3 chain | AAH02484, |
| | | | AAI50626 |
| MDDGA type | B3GNT1 gene | UDP-GlcNAc:betaGal beta-1,3- | |
| | | N-acetylglucosaminyltransferase | |
| | | 2 | |
| MDDGA1 type | POMT1 gene | protein O-mannosyltransferase 1 | BAX07485 |
| MDDGA2 type | POMT2 gene | protein O-mannosyltransferase 2 | AAF62558 |
| MDDGA8 type | GTDC2 gene | protein O-linked mannose N- | NP_116195 |
| | | acetylglucosaminyltransferase 2 | |
| | | (beta 1,4-) | |
| MDDGA10 type | TMEM5 gene | ribitol xylosyltransferase 1 | AAH02596 |
| MDDGA11 type | B3GALNT2 gene | beta-1,3-N- | AAH16974 |
| | | acetylgalactosaminyltransferase 2 | |
| MDDGA12 type | SGK196 gene | protein O-mannose kinase | AAI13704 |

Other

| | | | |
|---|---|---|---|
| Duchenne muscular | Dystrophin | Dystrophin | AAA53189 |
| dystrophy | | | |
| Becker muscular | Dystrophin | Dystrophin | AAA53189 |
| dystrophy | | | |
| Carriers of Duchenne or | Dystrophin | Dystrophin | AAA53189 |
| Becker MD | | | |
| Emery-Dreifuss | emerin, lamin A, lamin C | emerin, lamin A, lamin C | NP_000108, |
| Muscular Dystrophy | | | NP_001269554, |
| | | | NP_733821 |
| Facioscapulohumeral | Unknown; short DNA | | |
| Muscular Dystrophy | segment on Chromosome 4 | | |
| (FSH, FSHD) | | | |
| Myotonic Dystrophy | DMPK contains an | DM1 protein kinase | AAH26328 |
| (DM) 1 | abnormally expanded | | |
| | section | | |
| Myotonic Dystrophy | ZNF9 | CCHC-type zinc finger nucleic | AAR89462 |
| (DM) 2 | | acid binding protein | AAK91676 |
| Oculopharyngeal | PABPN1 | poly(A) binding protein nuclear 1 | AAH10939 |
| Muscular Dystrophy | | | |
| (OPMD) | | | |
| Friedreich's Ataxia (FA) | frataxin | frataxin | AAA98509 |
| Mitochondrial | genes involved in | | |
| Myopathies | mitochondrial disease | | |
| | normally make proteins that | | |
| | work inside the | | |
| | mitochondria | | |
| Mitochondrial | mitochondria are often | | |
| Myopathies | damaged as a result of | | |
| | aging | | |
| Mitochondrial | mitochondria are often | | AAA53189 |
| Myopathies | damaged as a result of | | |
| | cancer treatments | | |

Dominant Limb Girdle Muscular Dystrophy Subtype Numbers

| | | | |
|---|---|---|---|
| LGMD1A | MYOT mutation | myotilin | AAD44754 |
| LGMD1B | LMNA mutation | lamin A/C | NP_001269554, |
| | | | NP_733821 |
| LGMD1C | CAV3 mutation | caveolin 3 | CAA75042 |
| LGMD1D | DNAJB6 mutation | DnaJ heat shock protein family | CAG38529 |
| | | (Hsp40) member B6 | |
| LGMD1E | DES mutation | desmin | NP_001918 |
| LGMD1F | TNP03 mutation | transportin 3 | EAL24106 |

Recessive Limb Girdle Muscular Dystrophy Subtype Numbers

| | | | |
|---|---|---|---|
| LGMD2A | CAPN3 mutations | calpain 3 | AAD28253 |
| LGMD2B | DYSF mutations | dysferlin | AAC63519 |
| LGMD2C, also called | SGCG mutations | sarcoglycan gamma | NP_000222 |
| SCARMD1 | | | |
| LGMD2D, also called | SGCA mutations | sarcoglycan alpha | AAH25702 |
| SCARMD2 | | | |
| LGMD2E | SGCB mutations | sarcoglycan beta | CAG33091 |
| LGMD2F | SGCD mutations | sarcoglycan delta | AAH20740 |
| LGMD2G | TCAP mutations | titin-cap | NP_003664 |
| LGMD2H | TRIM32 mutations | tripartite motif containing 32 | NP_036342 |
| LGMD2I | FKRP mutations | fukutin related protein | NP_001034974 |
| LGMD2J | TTN mutations | titin | CAA62188 |
| LGMD2K | POMT1 mutations | protein O-mannosyltransferase 1 | BAX07485 |
| LGMD2L | ANO5 mutations | anoctamin 5 | NP_998764 |
| LGMD2M | FKTN mutations | fukutin | AAI17701 |
| LGMD2N | POMT2 mutations | protein O-mannosyltransferase 2 | AAF62558 |
| LGMD2O | POMGnT1 mutations | protein O-linked mannose N- | NP_001230695 |
| | | acetylglucosaminyltransferase 1 | |
| | | (beta 1,2-) | |
| LGMD2P | DAG1 mutations | dystroglycan 1 | NP_004384 |
| LGMD2Q | PLEC1 mutations | plectin | CAA91196 |
| LGMD2R | DES mutations | desmin | NP_001918 |
| LGMD2S | TRAPPC11 mutations | trafficking protein particle | NP_068761 |
| | | complex 11 | |
| LGMD2T | GMPPB mutations | GDP-mannose | NP_068806 |
| | | pyrophosphorylase B | |
| LGMD2U | ISPD mutations | CDP-L-ribitol pyrophosphorylase | NP_001094896 |
| | | A | XP_001126777 |
| | | | XP_001131763 |
| | | | XP_001132057 |
| LGMD2V | GAA mutations | glucosidase alpha, acid | XP_005257251 |
| LGMD2W | LIMS2 mutations | LIM zinc finger domain | AAM77350 |
| | | containing 2 | |
| LGMD2X | BVES mutations | blood vessel epicardial substance | AAH40502 |
| LGMD2Y | TOR1AIP1 mutations | torsin 1A interacting protein 1 | NP_001254507 |

Congenital Myopathies

| | | | |
|---|---|---|---|
| Centronuclear | deficiencies of | myotubularin | NP_000243 |
| myopathies, including | myotubularin | | |
| myotubular myopathy | | | |
| Central core | Defects of ryanodine | Ryanodine receptor or Ca | P21817 |
| disease/malignant | receptor and/or Ca handling | handling protein | |
| hyperthermia | protein | | |
| susceptibility | | | |
| Centronuclear | defects or deficiencies of | myotubularin | NP_000243 |
| myopathies, including | myotubularin | | |
| myotubular myopathy | | | |
| Nemaline myopathy (rod | | | |
| body disease) | | | |

Distal Muscular Dystrophies

| | | | |
|---|---|---|---|
| Finnish (tibial) distal | Titin | Titin | CAA62188 |
| myopathy | | | |
| Gowers-Laing distal | mutations in the MYH7 | myosin heavy chain 7 | NP_000248 |
| myopathy | gene, which instructs for | | XP_005267753 |
| | myosin heavy chain 7, a | | |
| | protein that participates in | | |
| | muscle contraction | | |
| Hereditary inclusion- | abnormal clumps of cellular | | |
| body myositis | material; and vacuoles, | | |
| (myopathy) type 1 | which are cellular bubbles. | | |
| (HIBM1) | The cause is unknown. | | |
| Miyoshi distal myopathy | Dysferlin | Dysferlin | AAC63519 |
| Nonaka distal myopathy | GNE gene | glucosamine (UDP-N-acetyl)-2- | NP_001121699 |
| (inclusion body | | epimerase/N-acetylmannosamine | |
| myopathy) | | kinase | |
| VCP Myopathy/ | VCP or p97 | valosin containing protein | AAI22551 |
| IBMPFD | | | |

Metabolic Myopathies

| | | | |
|---|---|---|---|
| Acid maltase deficiency | Acid alpha-glucosidase | Acid alpha-glucosidase | AAA52506 |
| (Type II GSD; Pompe | | | |
| disease) | | | |
| Carnitine deficiency | Primary - missing carnitine | | |
| | transporter; secondary - | | |
| | happens in other metabolic | | |
| | diseases | | |
| Carnitine palmityl | carnitine palmityl | carnitine palmityl | EAX06753 |
| transferase deficiency | transferase 2 enzyme (CPT2) | transferase 2 enzyme | |
| Debrancher enzyme | defect in the debrancher | debrancher enzyme | NP_000633 |
| deficiency (GDS Type | enzyme gene, which | | |
| III; Cori or Forbes | interferes with the | | |
| disease) | breakdown of glycogen | | |
| Lactate dehydrogenase | lactate dehydrogenase | lactate dehydrogenase | CAE11711 |
| deficiency | enzyme | enzyme | |
| Myoadenylate | Myoadenylate deaminase | Myoadenylate deaminase | NP_000027 |
| deaminase deficiency | deficiency | | |
| Phosphofructokinase | Phosphofructokinase | Phosphofructokinase | AAA91985 |
| deficiency (GSD VII; | deficiency | | |
| Tarui disease) | | | |
| Phosphoglycerate kinase | phosphoglycerate kinase | phosphoglycerate kinase | AAA60078 |
| deficiency | enzyme | enzyme | |
| Phosphoglycerate | phosphoglycerate mutase | phosphoglycerate mutase | AAA64238 |
| mutase deficiency | enzyme | enzyme | |
| Phosphorylase | phosphorylase (also known as | phosphorylase (also known as | NP_005600 |
| deficiency (GDS Type | myophosphorylase) enzyme | myophosphorylase) enzyme | |
| V; McArdle disease) | | | |
| Type IV - Anderson's | Glycogen branching enzyme | Glycogen branching enzyme | NP_000149 |
| Disease | | | |
| GSD X/GSD 10 | Phosphoglycerate mutase | Phosphoglycerate mutase | AAA64238 |
| GSD XII/GSD 12 | Aldolase A | Aldolase A | CAA30979 |
| GSD XIII/GSD 13 | β-enolase | β-enolase | NP_443739 |
| GSD XV/GSD 15 | Glycogenin-1 | Glycogenin-1 | AAD31084 |

Others

| | | | |
|---|---|---|---|
| Myoclonic epilepsy | CSTB, EPM2A, NHLRC1, | cystatin B; EPM2A glucan | NP_000091.1, |
| | mitochondrial disorders | phosphatase, laforin; NHL repeat | AAH05286.1, |
| | | containing E3 ubiquitin protein | NP_940988.2, |
| | | ligase 1 | mito replacement |
| Autosomal dominant | CSF1R | colony stimulating factor 1 | AAH47521.1 |
| adult-onset | | receptor | |
| leukodystrophy | | | |
| Epsilon | SGCE | sarcoglycan epsilon | AAQ89401.1 |
| sarcoglycanopathy | | | |
| Compton-North | CNTN1 | contactin 1 | AAH36569 |
| congenital myopathy | | | |
| Early onset myopathy, | MEGF10 | multiple EGF like domains 10 | AAH20198.1 |
| areflexia, respiratory | | | |
| distress, and dysphagia | | | |
| Hypertrophic | MYBPC3 | myosin binding protein C, cardiac | ABQ59032.1 |
| cardiomyopathy 4 | | | |
| Proximal myopathy and | MYH2 | myosin heavy chain 2 | AAH93082.1 |
| ophthalmoplegia | | | |
| Myotubular myopathy | MTM1 | myotubularin 1 | NP_000243.1 |
| Leukodystrophy, adult- | LMNB1 | Lamin B1 | NP_005564.1 |
| onset, autosomal | | | |
| dominant | | | |
| Neurofibromatosis Type | NF1 | neurofibromin | BAA02150.1 |
| 1 | | | |
| Methylmalonic acidemia | MUT | methylmalonyl-CoA mutase | ALQ33660.1 |
| Methylmalonic | MMACHC | Cobalamin C | AAH06122.3 |
| acidemia, | | | |
| Homocysteinuria | | | |
| Epilepsy, progressive | LMNB2 | Lamin B2 | NP_116126.3 |
| myoclonic, 9 | | | |
| Friedreich's Ataxia | FXN | Frataxin | AAH23633.1 |
| Meconium ileus in cystic | CFM1 | Cystic Fibrosis Modifier 1 | |
| fibrosis | | | |
| Tay-Sachs | HEXA | β-Hexosaminidase A | AAB00965.1 |
| Left ventricular | DTNA | Dystrobrevin alpha | AAH05300.1 |
| noncompaction | | | |
| Left ventricular | DTNB | Dystrobrevin beta | XP_024308511.1 |
| noncompaction | | | |
| Epidermolysis bullosa, | DST | Dystonin | AAH65536.1 |
| Neuropathy, hereditary | | | |
| sensory and autonomic, | | | |
| type VI | | | |

Nemaline Myopathies

| | | |
|---|---|---|
| NEB | Nebulin | NP_001157979.1 |
| ACTA1 | actin alpha 1, skeletal muscle | CAG38754.1 |
| TPM3 | Tropomyosin 3 | AAH62740.1 |
| TPM2 | Tropomyosin 2 | EAW58358.1 |
| TNNT1 | Troponin TI, slow skeletal muscle | AAI07799.1 |
| CFL2 | Cofilin 2 | AAF97934.1 |
| KBTBD13 | kelch repeat and BTB domain | NP_001094832.1 |
| | containing 13 | |
| KLHL40 | kelch like family member 40 | NP_689606.2 |
| KLHL41 | kelch like family member 41 | NP_006054.2 |
| LMOD3 | leiomodin 3 | AAH39202.1 |

Centronuclear myopathies

| | | |
|---|---|---|
| MTM1 | myotubularin 1 | NP_000243.1 |
| BIN1 | bridging integrator 1 | NP_647601.1 |
| RYR1 | ryanodine receptor 1 | P21817.3 |
| TTN | titin | XP_016860308.1 |
| DNM2 | dynamin 2 | AAH39596.1 |
| MYF6 | myogenic factor 6 | CAG46563.1 |
| MTMR14 | myotubularin related protein 14 | NP_001070993.1 |

Hypertrophic cardiomyopathy

| | | |
|---|---|---|
| MYH7 | myosin heavy chain 7 | NP_000248.2 |
| MYBPC3 | myosin binding protein C, cardiac | ABQ59032.1 |
| TNNT2 | troponin T2, cardiac type | AAK92231.1 |
| TNNI3 | troponin I3, cardiac type | CAG46782.1 |

Malignant hyperthermia susceptibility

| | | | |
|---|---|---|---|
| Type 1 | RYR1 | ryanodine receptor 1 | P21817.3 |
| Type 5 | CACNA1S | calcium voltage-gated channel | AAI33672.1 |
| | | subunit alpha1 S | |
| Type 2 | unknown | | |
| Typc 3 | CA2D1 | voltage-dependent calcium | P54289 |
| | | channel subunit alpha-2/delta-1 | |
| | | precursor | |
| Type 4 | unknown | | |
| Type 6 | unknown | | |

Dilated cardiomyopathy

| | | |
|---|---|---|
| LDB3 | LIM domain binding 3 | ALQ34003.1 |
| MYOZ2 | myozenin 2 | NP_057683.1 |
| CALR3 | calreticulin 3 | EAW84546.1 |
| TBX20 | T-box transcription factor 20 | NP_001071121.1 |
| COA6 | cytochrome c oxidase assembly | NP_001193570.2 |
| | factor 6 | |
| MYL3 | myosin light chain 3 | NP_000249.1 |
| MYL2 | myosin light chain 2 | CAG33243.1 |
| LAMP2 | lysosomal associated membrane | AAB67314.1 |
| | protein 2 | |
| FKTN | fukutin | AAI17701.1 |
| MT-TL1 | mitochondrially encoded tRNA | |
| | leucine 1 | |
| MYH6 | myosin heavy chain 6 | NP_002462.2 |
| DSP | desmoplakin | NP_001305963.1 |
| DNAJC19 | DnaJ heat shock protein family | NP_660304.1 |
| | (Hsp40) member C19 | |
| DOLK | dolichol kinase | NP_055723.1 |
| ABCC9 | ATP binding cassette subfamily | AAH33804.1 |
| | C member 9 | |
| EYA4 | EYA transcriptional coactivator | CAA76636.1 |
| | and phosphatase 4 | |
| PDCD1 | programmed cell death 1 | AAO63583.1 |
| PLN | phospholamban | NP_002658.1 |
| TAZ | tafazzin | NP_000107.1 |
| NEXN | nexilin F-actin binding protein | AAI11396.1 |
| GATAD1 | GATA zinc finger domain | AAH31091.1 |
| | containing 1 | |
| CSRP3 | cysteine and glycine rich protein | AAH24010.1 |
| | 3 | |
| RBM20 | RNA binding motif protein 20 | NP_001127835.2 |
| VCL | vinculin | NP_003364.1 |
| HSD17B10 | hydroxysteroid 17-beta | ALQ33565.1 |
| | dehydrogenase 10 | |
| JUP | junction plakoglobin | AAH11865.1 |
| MT-TV | mitochondrially encoded tRNA | |
| | valine | |
| RAF1 | Raf-1 proto-oncogene, | NP_001341618.1 |
| | serine/threonine kinase | |
| ACE | angiotensin I converting enzyme | EAW94319.1 |
| IDH2 | isocitrate dehydrogenase | NP_002159.2 |
| | (NADP(+)) 2, mitochondrial | |
| MT-TK | mitochondrially encoded tRNA | |
| | lysine | |
| RYR2 | ryanodine receptor 2 | NP_001026.2 |
| GLB1 | galactosidase beta 1 | AAH07493.1 |
| MLYCD | malonyl-CoA decarboxylase | AAH00286.1 |
| APOA1 | apolipoprotein A1 | AAS68227.1 |
| TMEM70 | transmembrane protein 70 | EAW87022.1 |
| SMAD4 | SMAD family member 4 | AAH02379.1 |
| CAV3 | caveolin 3 | NP_203123.1 |
| PRKAG2 | protein kinase AMP-activated | NP_057287.2 |
| | non-catalytic subunit gamma 2 | |
| PKP2 | plakophilin 2 | ACD13293.1 |
| CPS1 | carbamoyl-phosphate synthase 1 | CAA75785.1 |
| MYH7 | myosin heavy chain 7 | NP_000248.2 |
| TPM1 | tropomyosin 1 | NP_001018005.1 |
| MT-TH | mitochondrially encoded tRNA | |
| | histidine | |
| DSC2 | desmocollin 2 | AAH63291.1 |
| NDUFB9 | NADH:ubiquinone | NP_004996.1 |
| | oxidoreductase subunit B9 | |
| NDUFV2 | NADH:ubiquinone | AAH17487.1 |
| | oxidoreductase core subunit V2 | |
| TMEM43 | transmembrane protein 43 | NP_077310.1 |
| SOD2 | superoxide dismutase 2 | NP_001309743.1 |
| MYBPC3 | myosin binding protein C, cardiac | NP_000247.2 |
| SLC25A4 | solute carrier family 25 member 4 | AAH61589.1 |

In some embodiments, the cells have been modified to contain a higher level of the one or more therapeutic agents than unmodified cells. In some embodiments, the one or more therapeutic agents or a nucleic acid encoding the one or more therapeutic agents have been introduced into the cells, e.g., by transfection, transduction, infection, electroporation, or any combination thereof. In some embodiments, the cells have been modified to produce an expression vector, e.g., a plasmid vector or a viral vector. Viral vectors that can be used include, but are not limited to, retrovirus, lentivirus, adeno-associated virus, poxvirus, alphavirus, baculovirus, vaccinia virus, herpes virus, Epstein-Barr virus, and adenovirus vectors. Non-viral vectors include plasmids, liposomes, electrically charged lipids (cytofectins), nucleic acid-protein complexes, and biopolymers.

In some embodiments, the EVs are obtained from HEK293 cells. HEK293 cells have been determined to produce EVs comprising a variety of components that can be used as therapeutic agents. In an embodiment, the one or more therapeutic agents is involved in a biological process selected from mitotic cell cycle process; mitotic cell cycle; cellular component biogenesis; intracellular transport; multi-organism cellular process; symbiosis, mutualism through parasitism; interspecies interaction between organisms; viral process; mRNA metabolic process; and RNA processing.

The EVs of the compositions are cell-derived nanoparticles between about 30 nm and 1000 nm. In an embodiment, the EVs are small EVs (sEVs, <200 nm) or medium/large EVs (m/l EVs >200 nm), and may be measured by modal size. In an embodiment, the EVs are between about 30 to 250 nm, about 30 to 200 nm, between about 50 to 180 nm, or between about 100 to 170 nm. In an embodiment, the EV may be 30, 40, 50, 60, 70 80, 90, 100, 110, 120, 130, 140, 150, 160, 170, 180, 190, 200, 210, 220, 230, 240, or 250 nm in size. EVs can also be subcategorized by their densities or by biochemical analyses identifying protein markers such as the cluster of differentiation (CD) proteins (CD9, CD63, CD81, etc.), syntenin-1, annexin A-5, ALIX. Théry, et al., J Extracell Vesicles. 7(1), 1535750 (2018).

### Methods

One aspect of the invention relates to the use of EVs as delivery vehicles for therapeutic agents. In some embodiments, the EVs may naturally contain the therapeutic agents. In other embodiments, the cells from which the EVs are derived may be modified to contain the therapeutic agents or higher levels of the therapeutic agent, which then end up in the EVs. The use of EVs to deliver therapeutic agents such as viral vectors that may raise an immune response may prevent or diminish the immune response against the vector. Additionally, EVs can cross most membranes, including the blood brain barrier, so they may be used to deliver therapeutic agents to the brain as well as other tissues and organs. Another advantage is the ability of EVs to rapidly deliver therapeutic agents to a tissue, e.g., within 24 hours.

Without being bound by theory, EVs of the present invention can deliver cargo by transfer of cargo into recipient cells by a variety of processes. In some embodiments, once released into the extracellular milieu, EVs deliver their cargo into recipient cells via numerous mechanisms, including direct membrane fusion, macro- and micro-pinocytosis, or endocytosis mediated by membrane receptors, membrane rafts, clathrin and caveolin. Transmembrane proteins on EVs act as ligands to protein receptors on recipient cell types, thus increasing the chance of an EV being taken up by the recipient cell via one of the previously mentioned mechanisms. Through these mechanisms, the molecular cargo of EVs can be transferred into the recipient cells where it can directly engage in signaling pathways (proteins and mRNA) or modulate gene expression (non-coding RNA species).

### Muscular Pathologies

The EV compositions of the present invention can be used in the treatment of muscular pathologies. Thus, one aspect of the invention relates to a method of treating a muscle-related disorder, condition, or damage in a subject in need thereof, comprising administering a therapeutically effective amount of the EVs to the subject; thereby treating the muscle-related disorder, condition, or damage. In some embodiments, the disorder, condition, or damage is a cardiac muscle-related disorder, condition, or damage.

Exemplary muscular diseases, disorders or conditions can include primary or secondary muscular pathologies. Primary congenital muscular pathologies, such as the muscular dystrophies, occur at a rate of 1-10 per 100,000 people in the population. In Duchenne Muscular Dystrophy (DMD), the most severe of these diseases, there is a median life expectancy of ˜29.9 years with ventilatory support. Crisafulli, et al. Orphanetj rare dis. 15(1), 1-141 (2020). This disease is attributed by mutations in the dystrophin gene, resulting in a shift in the reading frame and subsequently no production of the dystrophin protein. Dystrophin functions as a scaffolding protein for the dystrophin associated glycoprotein complex (DAGC), and connects it to the extracellular matrix, and cytoskeletal proteins, such as actin. Other mutations, such as those involved in Becker Muscular Dystrophy, result in the production of a truncated form of the dystrophin protein and thus have a less severe phenotype and improved life expectancy. Muscular pathologies may also present as secondary or acquired conditions as a comorbidity of some of the most prevalent diseases facing modern society, such as cancer, acquired immunodeficiency syndrome, rheumatoid arthritis, chronic obstructive pulmonary disease, as well as heart and renal failure. Muscle degeneration is also associated with other conditions, such as aging (sarcopenia) and anorexia. The processes underlying muscle degeneration in these conditions commonly involve chronic inflammatory signaling and metabolic resistance to anabolic signaling promoting a chronic catabolic state in the musculature, with common inflammatory modulators in these conditions include tumor necrosis factor-α (TNF-α) and interleukins-1, -6 and -8; thus, these modulators can be used as therapeutic agents in the present invention. Muscular pathologies encompass inherited disorders arising from genetic mutations in genes encoding proteins that are critical to muscle function to acquired secondary pathologies as comorbidities of other conditions. Exemplary muscle disorders, diseases or conditions include those identified in Table 2.

Thus, one aspect of the invention relates to methods of making the compositions that are useful for targeting muscle cells and delivery of therapeutic agents. In an embodiment, a method of making the composition comprises introducing a nucleic acid encoding the targeting membrane protein, or fragment thereof, into the cell; culturing the cells in cell culture medium to thereby express the target membrane protein; and isolating the composition comprising the extracellular vesicles from the cell culture medium. In some embodiments, the EVs comprise a muscle targeting protein from Table 1 introduced into the cell that allows for myotropic delivery of the EVs.

The nucleic acid can be introduced into the cells by transfection, transduction, infection, electroporation, or any combination thereof. In some embodiments, the EVs are isolated from cells that have been modified to contain a higher level of the one or more therapeutic agents than unmodified cells. In some embodiments, the one or more therapeutic agents or a nucleic acid encoding the one or more therapeutic agents or vectors (e.g., viral vectors) delivering a nucleic acid encoding one or more therapeutic agents have been introduced into the cells.

Having described the present invention, the same will be explained in greater detail in the following examples, which are included herein for illustration purposes only, and which are not intended to be limiting to the invention.

### EXAMPLES

### Example 1

Applicants describe evaluation of muscle-targeted EVs using native muscle-enriched membrane proteins in this example. By engineering EVs to display targeting moieties to particular tissue/cell types, they may be utilized as biocompatible, targeted drug delivery vehicles. Although this strategy has the potential to overcome many of the limitations of current vectors for therapeutics used to treat muscular pathologies, such as AAV, very few studies have investigated the implementation of engineered EVs for the targeted delivery of therapeutics to skeletal and cardiac muscle. The only documented attempt ultimately ended in failure in vivo. Wood, et al., Nat biotech. 29(4), 341-345 (2011). However, advancements in EV-scaffolding proteins for enhanced engineering could be a promising strategy for generating improved myotropic EV formulations. Additionally, a number of proteins expressed in muscle stem cells and myoblasts possess critical functions in myogenic fusion and adherence. If expressed in the EV membrane, these proteins could feasibly impart these myotropic properties on the EVs. Thus, more work is needed to fully elucidate the potential of these novel myotropic EVs to deliver therapeutic cargoes in a targeted manner.

The strategies for muscle-targeted delivery of therapeutics attempted thus far have largely focused on engineering synthetic myotropic peptides (MPs), ASSLNIA (SEQ ID NO:1) and RRQPPRSISSHP (SEQ ID NO:2), either directly to the therapeutic (ASO) or the vector used for delivery (AAV2 or EV). However, many native muscle-enriched membrane proteins exist. The proteins MyoMaker (MYMK) and MyoMixer (MYMX) are myogenic membrane-fusion proteins involved in the fusion of myoblasts into muscle fibers during embryonic development and repair of adult muscle. It was found that when the genes encoding either of these proteins were deleted, complete fusion of myoblasts into myotubes was inhibited, emphasizing their roles in muscle-specific membrane fusion. Further, when over-expressed via transfection in fibroblasts, MYMK enabled the fusion of the fibroblasts to myotubes in vitro. In addition, M-Cadherin (M-Cad) is a membrane protein that adheres quiescent satellite cells to adjacent muscle fibers. Given the physiologic roles of these proteins in membrane fusion/adherence to the muscle fiber membrane and the sparsity of myotropic EV candidates, Applicant examined the myotropic properties of EVs expressing each of these native myotropic protein candidates. Preliminary experiments screening these myotropic EV candidates identified the MYMK-EV formulation as showing the greatest myotropic potential in vitro. Applicant assessed this by measuring the delivery of EV-derived protein cargo into C2C12 myotubes in vitro, a cell-type that is commonly used for these types of experiments. In the current study, the myotropic properties of EVs displaying the native myotropic protein candidate, MYMK, in vivo was examined for affinity for skeletal muscle relative to non-engineered Human Embryonic Kidney (HEK293) cell-derived EVs in both C57/BL6 and mdx (DMD, exon 23 nonsense point mutation) mouse models.

### Screening of Native Myotropic Protein Candidates

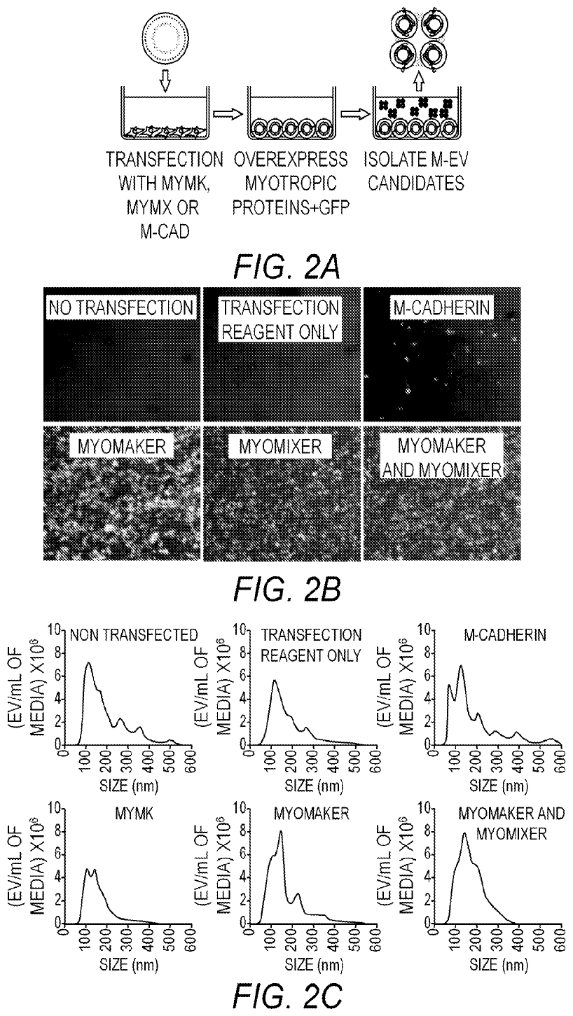

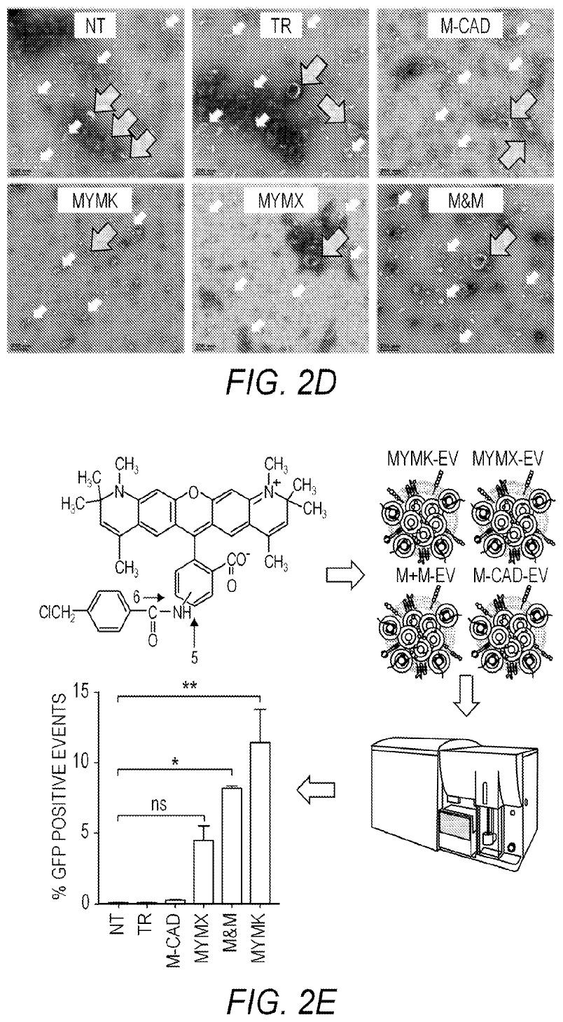

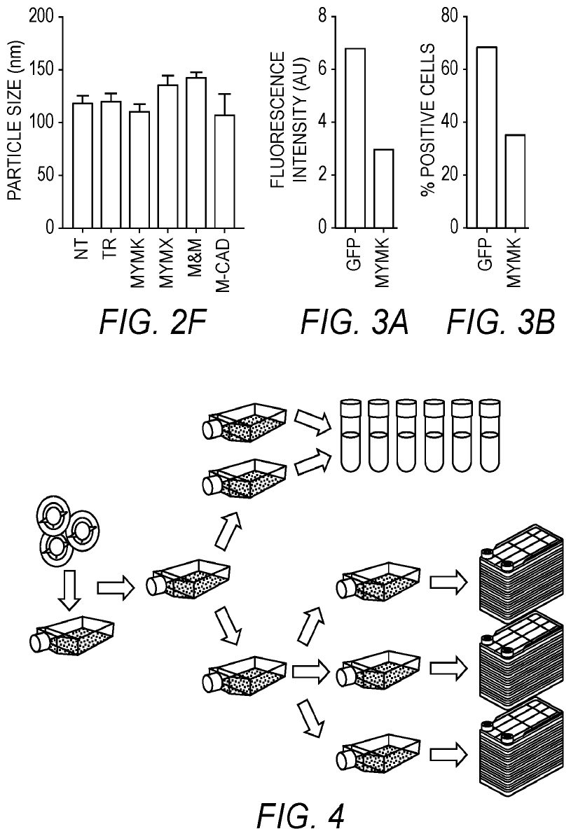

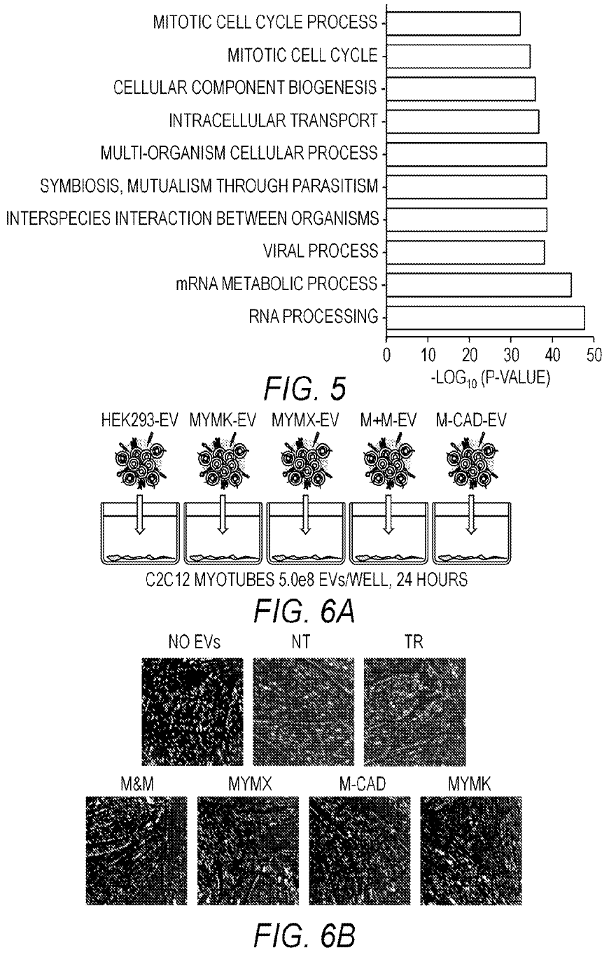

Following the initial transient transfection of the MYMK, MYMX and M-Cad plasmids, the cells displayed varying levels of recombinant protein expression, measured using confocal microscopy to analyze the signal from the GFP tag associated with each protein (FIGS. 2A, 2B). Following the transfections, NTA, which measures all particles capable of scattering light to produce a detectable signal, was used to examine the particle concentration and size distribution of the EVs produced. Cells receiving only the transfection reagent (TR) and the cells transfected with MYMK released significantly less EVs than the other groups (p<0.05; FIG. 2C); however, there were no differences in the modal size of the EVs produced in each group with an average modal size of 122.8 nm (p=0.1885) which fell in the range typically associated with EVs (FIG. 2C). Transmission electron microscopy (TEM) was used to qualitatively assess the EVs produced in each group. The TEM results provide representative data suggesting that EVs make up a very small fraction of the total biomolecular constituents in the conditioned culture medium when using precipitation as the purification method, especially in the presence of FBS. This is evidenced by the abundance of serum-derived lipoproteins and protein aggregates observed in the TEM images, which tend to make up a large proportion of biomolecular constituents in each image (FIG. 2D). Given these initial results in the screening experiments, serum-free OptiMEM medium was used as the HEK293 EV production medium for all subsequent experiments, which is consistent with others for this cell-type. Additionally, using flow cytometry, the fluorescence intensity of the endogenous GFP-tag associated with the recombinant proteins of each M-EV formulation was measured. The ubiquitous EV luminal protein counterstain, ExoGlow-Red, was used to calculate the percentage of total EVs containing the myotropic proteins. Both MYMK and the MYMK+MYMX (M&M) groups showed a significant level of recombinant protein incorporation into the EVs relative to the EVs from non-transfected HEK293 cells (NT) (MYMK, p=0.002; M&M, p=0.0124; FIG. 2E). However, the M&M group showed lower protein incorporation overall.

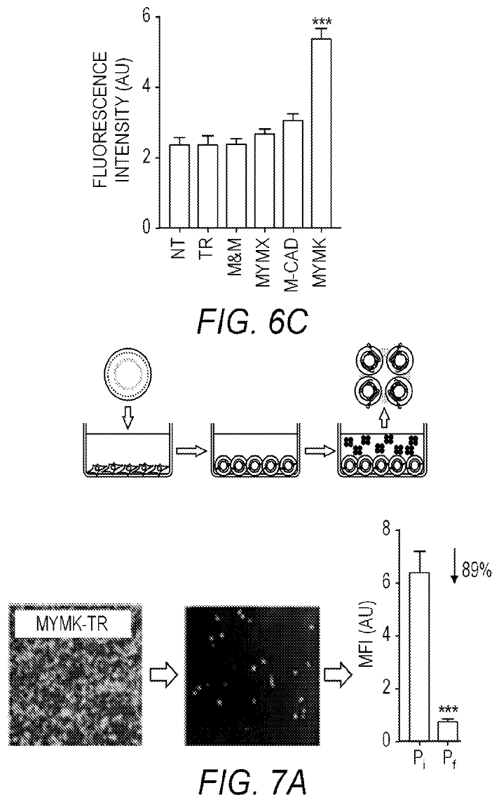

To analyze the myotropic properties of the native MT-EV candidates, the luminal protein cargo of each formulation was labeled with the amine-reactive probe, CFDA-SE, and incubated with C2C12 myotubes in vitro. Free dye unbound to EVs post-labeling was removed. The fluorescent signal from the labeled EV cargo delivered into the myotubes was measured using fluorescence microscopy. The MYMK-EVs were the only group to show a significant difference in delivered protein cargo with a 125% increase in the fluorescently-labeled protein cargo delivered into the myotubes compared to control EVs produced by non-transfected (NT) HEK293 cells (p=0.0005; FIGS. 6A-6C).

### Stable Producer Cell Line Generation

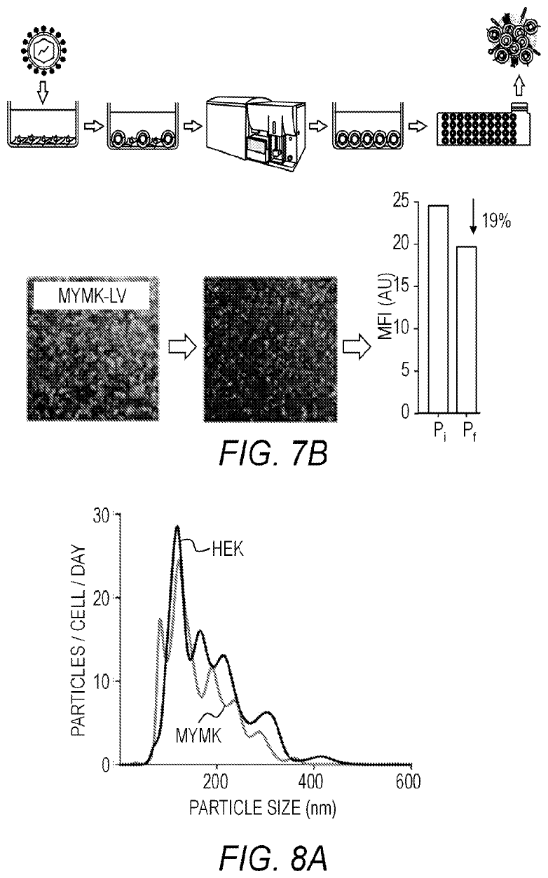

The in vitro experiments identified the MYMK-EV formulation as the most promising native M-EV construct. The next step was to analyze the myotropic properties of MYMK-EVs in vivo. This required much larger quantities of EVs than previously needed in the early in vitro experiments. It was estimated that a total of 2.9-3.8e12 EVs would be needed to dose the 3-4 mice per experimental group for the eight experimental groups. In order to produce this quantity of EVs, a stable cell line was needed to scale up into larger cell culture vessels to produce the needed quantities. The transfection efficiency of the cationic lipid reagent jetOPTIMUS was compared to lentiviral vectors containing a MYMK-encoding plasmid. Following selection of the GFP positive cells using fluorescence assisted cell sorting, cells transfected with either method were passaged ten times. Over the course of these passages, the cells transfected with the cationic lipid reagent displayed a dramatic 89% decrease in fluorescence signal (FIG. 7A) while the cells transfected using the lentiviral vectors showed a more modest 19% decline in signal (FIG. 7B). This coincides with the previous literature suggesting that cationic lipid-based reagents result in a much less efficient incorporation of the transgene into the recipient cells' genomes via homologous recombination as opposed to lentiviral transduction of the transgene. Thus, the MYMK-EV producer HEK293 line was generated using the lentiviral transduction method. The stably transfected cells were successfully seeded into a 6-well plate and then scaled to 875 cm² flasks for MYMK-EV production.

### EV Characterization

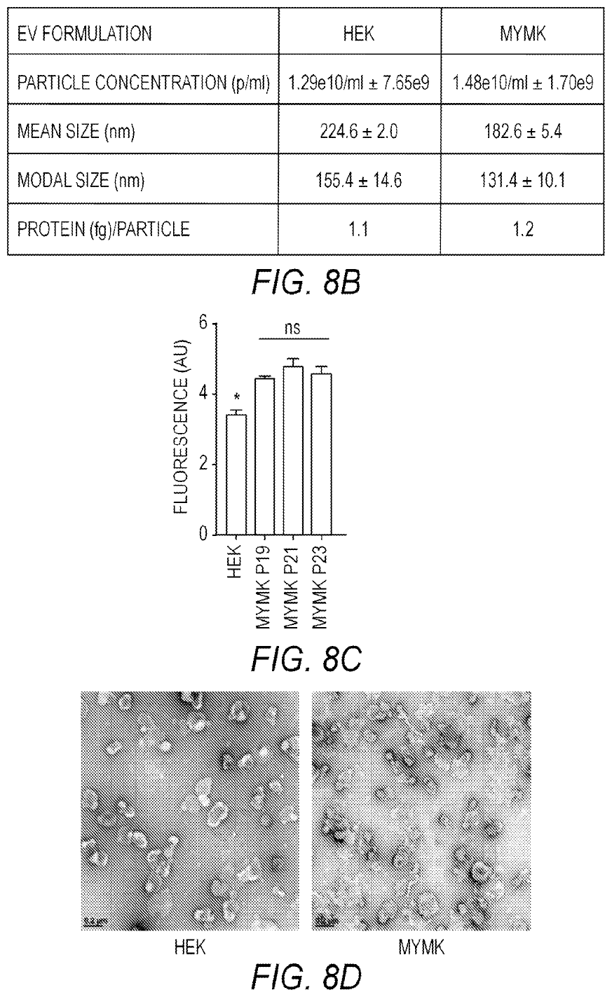

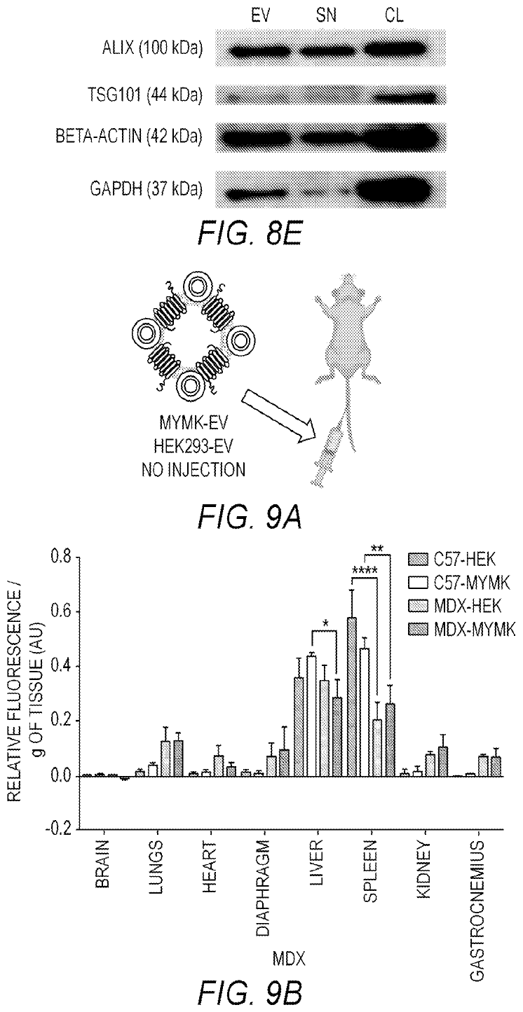

Once a stable MYMK-EV producer cell line was established the EVs produced were analyzed using NTA, TEM and western blot as per the MISEV guidelines (FIGS. 8A-8E). The NTA revealed particles in the typical size range of EVs with a modal size of ˜155.4 nm for EVs from non-transfected HEK293 cells and ˜131.4 nm from the MYMK-EV producer cells (FIGS. 8A, 8B). The producer cells and EVs were also analyzed using a fluorescent plate reader to track MYMK expression/contents over 6 passages during EV production (FIG. 8C). This revealed no significant change in the expression of MYMK in the cells over this timeframe; however, the EVs could not be detected using this method. TEM was used to analyze the morphology of the EVs produced by each of these cell lines. Both produced particles in a similar size range (˜50-200 nm) as demonstrated by the NTA data with the typical EV cup-shaped morphology. However, in addition to the typical EV-like particles, TEM revealed additional debris that co-precipitated with the EVs during ultracentrifugation (FIG. 8D). EV samples were probed for the canonical EV-enriched protein markers, ALIX and TSG101 as well as cellular markers, beta actin and GAPDH, that are not typically enriched in EVs. Western blot analysis confirmed the presence of the EV markers in the UC pellet sample; however, it also revealed the pellet to be positive for beta actin and GAPDH. This confirmed the previous suspicions that cellular debris made it through the clarification process and into the UC pellet sample. In addition, ALIX and TSG101 were also identified in the supernatant following UC, suggesting that these proteins may be secreted independent of EVs as well (FIG. 8E).

### Biodistribution of EVs Using a Lipophilic Dye

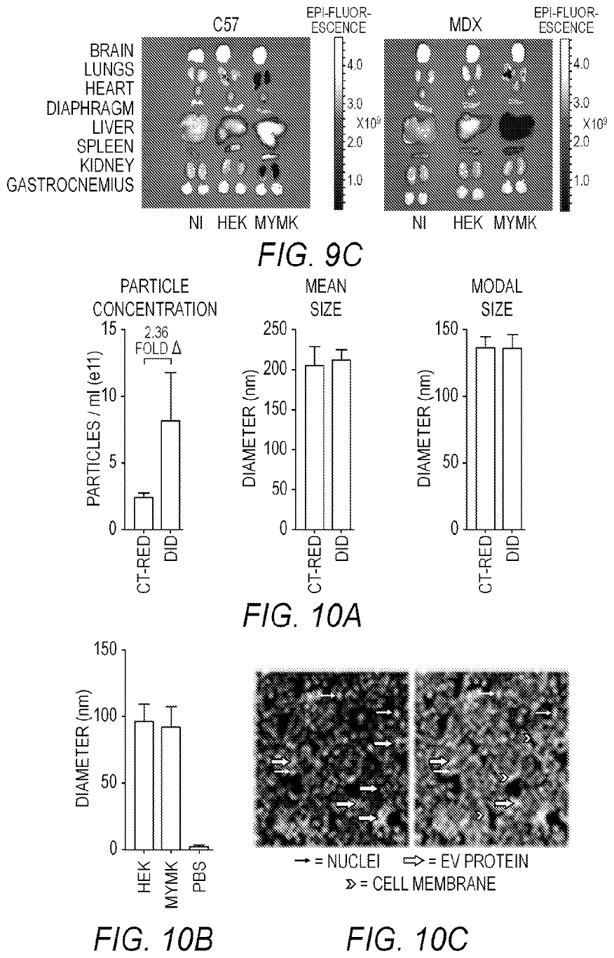

DiD-labeled HEK and MYMK-EVs were administered to C57 or mdx mice via tail vein injection (FIG. 9A). The organs were harvested 24 h later and immediately imaged on an IVIS (FIG. 9C), after which they were homogenized and the EV-derived fluorescent signal was analyzed using a fluorescent plate reader (FIG. 9B). No significant differences were observed between the biodistribution of the MYMK-EVs and non-engineered HEK293-EVs in the C57 or mdx mice with the majority of signal derived from each formulation predominantly localizing to the spleen and the liver. However, when comparing the biodistribution of the EVs in C57 to mdx mice, there was a significant decrease in fluorescence signal detected in the spleen for both EV types. Specifically, a 64% (p<0.0001) decrease in fluorescent signal was observed in the spleens of mdx mice relative to C57 following administration of the HEK-EV formulation and a 41% decrease (p=0.004) following administration of the MYMK-EV formulation. Interestingly, as the signal derived from the spleen decreased when comparing C57 to mdx mice, a nonsignificant increase in signal derived from many of the peripheral organs was also observed corresponding to the decreased signal in the spleen, potentially suggesting redistribution of the EVs in the mdx mice as compared to the C57 mice. Additionally, there was a 34% decrease (p=0.04) in fluorescent signal in the liver of mdx mice that received the MYMK-EV formulation as compared to that in the liver of C57 mice that received the same EVs, but this was not observed in the mice that received the HEK293-EV formulation.

### Biodistribution of EVs Using an Amine-Reactive Dye

Following the biodistribution experiments using the lipophilic probe, Applicant next assessed the biodistribution of the MYMK-EVs labeled with CT-Red. The proposed mechanism by which this class of dye works is by permeating the phospholipid bilayer of the EV followed by cleavage by intracellular esterases packaged within the EV. This transformation renders the dye impermeable to the phospholipid bilayer, allowing it to interact with any exposed amine groups within the EV. The amine-reactive dye was chosen in addition to the lipophilic dye to enable tracking of the intracellular delivery of EV-derived protein cargo into recipient tissues.

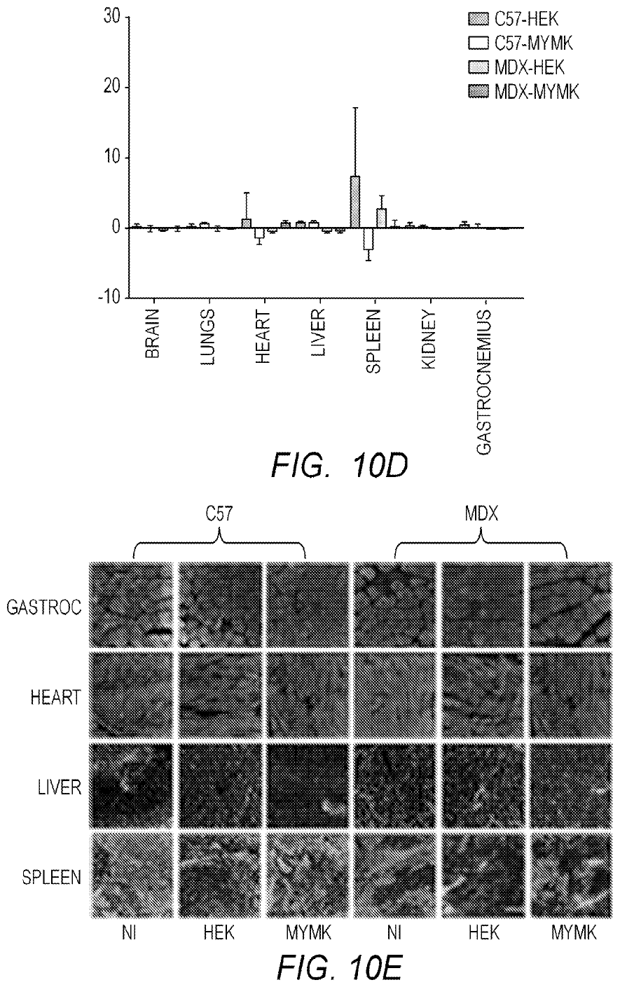

To dose the EVs for these experiments, a fluorescent plate reader was used rather than NTA. When analyzing batches of EVs derived from the same flasks of producer cells, it became evident that the measured particle count using NTA varied depending on which dye was used (FIG. 10A). Specifically, the sample stained with CT-Red had a concentration of 2.44e11 particles/ml whereas the sample stained with DiD had a concentration of 8.21e11 particles/ml; however, there was no effect on particle size. To minimize this variability, the EV doses were selected based on the fluorescent signal derived from the labeled protein in each sample rather than particle count (FIG. 10B).

HEK and MYMK-EVs were injected into both C57 and mdx mice at equivalent fluorescence intensities. Neither the fluorescent plate reader or IHC/imaging were able to reliably identify an EV-derived protein signal in any of the examined tissues above the autofluorescence of the tissue itself (FIG. 10D). A follow-up analysis of the EVs ability to deliver cargo into HEK293 cells in vitro confirmed the successful labeling of the EVs (FIG. 10C), indicating the EVs either contained inadequate protein cargo to be imaged in vivo at the given dose, or the labeled protein cargo may have been degraded within the 24 hour timespan following administration.

### Discussion

In the current study, a panel of native proteins with myotropic properties was examined in vivo for their ability to promote enhanced delivery of EV-derived cargoes into skeletal muscle.

MyoMaker (MYMK) and MyoMixer proteins were chosen due their roles in promoting myoblast fusion to skeletal muscle fibers following injury as well as the protein M-Cadherin, due to its role in adhering quiescent satellite cells to skeletal muscle fibers. Chen, Cell mol life sci. 77(8), 1551-1569 (2020); Bi, et al., Science. 356(6335), 323 (2017). To generate EVs displaying these candidate proteins, each protein was expressed via cationic lipid transfection in HEK293 cells and EVs harvested from the cell culture supernatant. The early results from conducting uptake experiments in C2C12 myotubes in vitro identified the MYMK-EV formulation as the most promising candidate with a ˜125% increase in protein delivery into C2C12.

Given these early findings, a stable MYMK-expressing cell line was generated and the MYMK-EVs administered to C57 and mdx mice in vivo with two different labeling methods. Overall using the lipophilic dye, DiD, a typical biodistribution pattern for both EV formulations was observed, with the majority of the fluorescent signal originating from the liver and spleen. The primary outcome observed was a significant decrease in the fluorescent signal in the spleen in MDX relative to C57 mice, regardless of the EV formulation administered. Simultaneous with this decrease was a non-significant increase in each of the peripheral tissues, except for the brain. In addition, the only observed difference in the biodistribution of the HEK293 and MYMK-EVs was a significant decrease in the MYMK-EV signal in the liver of mdx mice relative to that of the HEK293-EVs.

Although the MYMK-EV formulation yielded promising results in vitro, there was no observable difference in the delivery of these EVs to the skeletal muscle in vivo relative to the non-engineered HEK293 EVs. This is a similar outcome to that of Wood et al., Nat biotech. 29(4), 341-345 (2011), who investigated the biodistribution of EVs displaying the myotropic peptide, ASSLNIA (SEQ ID NO:1), fused to Lamp2b. The in vivo intramuscular niche is a more complex environment than myotubes in vitro, as it includes an endothelium and extracellular matrix that the EVs must first traverse before reaching the target muscle fibers. Given that the modal size of EVs is typically ˜100-200 nm and the EVs utilized in the current study were measured to have a modal size of ˜151 nm (HEK-EV) and ˜134 nm (MYMK-EV), it is possible that EVs were unable to traverse this endothelial barrier as efficiently. A biodistribution study of gold NPs that were 15, 50, 100 and 200 nm in diameter demonstrated that the larger sized NPs, in the modal size range of EVs, predominantly localized to the liver and spleen, whereas the smaller NPs had a longer circulation time and had improved distribution to the periphery. Skotland, et al., Adv drug deliv rev. 186, 114326 (2022). This suggests the size of drug carrier plays a large role in its biodistribution and EVs ˜100-200 nm modal size as in the current study may reduce the accessibility of EVs to tissues perfused by capillaries with a continuous endothelium. In the current study, the proportion of EV-derived fluorescent signal retained in the tissue normalized by organ weight was calculated in both C57 and mdx mice. Applicant found that ˜0.3% of the MYMK-EVs and 0.04% of the HEK-EVs were retained in the skeletal muscle tissue (gastrocnemius) of the C57 mice. These data represent a 6.5-fold increase in the signal observed in the skeletal muscle, although not statistically significant as the majority of the EV-derived signal was localized to the spleen (MYMK: ˜47%, HEK: ˜58%) and liver (MYMK: ˜44%, HEK: ˜36%). However, it should be noted that, in the case of DMD, even modest improvements in delivery may have a clinically relevant effect on dystrophin expression and muscle functionality.

Interestingly, in mdx mice the signal in the spleen showed a significant decline in EV-derived signal for both MYMK and HEK-EVs. Specifically, in the mdx mice the spleen accounted for ˜27% and ˜21% of the EV-derived fluorescent signal in the MYMK and HEK-EV groups, respectively. The MYMK-EVs also showed a significant decrease in the liver of mdx mice relative to C57 mice, which was not observed for the HEK-EVs. Simultaneously with the decrease in signal in the spleen, Applicant observed a non-significant increase in EV-derived signal in all peripheral tissues except for the brain. The gastrocnemius showed the greatest increase relative to the C57 mice, accounting for 7.2% (23-fold) of the EV-derived signal in the MYMK-EV group and 7.3% (181.5-fold) of the signal in the HEK-EV group. Overall, these data show a redistribution of the injected EV cargo away from the spleen and liver toward the peripheral tissues, with the greatest increase noted in the skeletal muscle.

Given the decreased signal in the spleen and increased signal in the skeletal muscle, and to a lesser extent the other peripheral tissues, of the mdx mice, it appears that the immune system could have a role in facilitating the redistribution of intravenously administered EVs. Driedonks et al., J Extracell Biol. 1(10), e59 (2021) noted a robust uptake of intravenously-administered EVs by circulating immune cells in Macaques. Thus, it is possible that under homeostatic conditions these cells largely accumulate in the spleen, but under chronic inflammation they may infiltrate the inflamed tissues in the periphery; the current study did not address the mechanism of biodistribution. Alternatively, blood vessel dysfunction in DMD may be another factor affecting the biodistribution of EVs in the mdx mouse. In DMD, it is thought that blood vessels may undergo degeneration and therefore have increased permeability. This could theoretically allow larger-sized particles such as EVs to access the musculature more easily. However, the mdx mice used in this study were relatively young (6-11 weeks), therefore, blood vessel degeneration was most likely not a significant factor. Thus, the inflammatory state of the peripheral tissues and immune cell migration may provide a better explanation of the results observed herein.

Another finding from these experiments was a discrepancy noted in the particle concentration yielded from NTA when staining the EV samples with DiD as compared to CT-Red. The DiD-labeled EV samples exhibited a 2.36-fold increase in particle concentration relative to the CT-Red samples. Given these discrepancies in particle count, a fluorescent plate reader was used to dose the EVs, which ensures that equal amounts of labeled material are administered. Given these findings and the variability associated with NTA, measuring the fluorescent signal in each sample to choose a dosage appears to be essential for attaining consistent results. Thus, although the protein-derived signal was not detectable in any of the tissues, these experiments have provided a wealth of practical insights into the dyes, dosing and methods of detection utilized in EV biodistribution studies.

Applicant examined the in vivo biodistribution and in vitro cell-type affinity of various novel myotropic EV candidates. In the in vivo biodistribution study, the biodistribution of EVs expressing the myogenic fusion protein, TMEM8C (MyoMaker, MYMK) was examined as compared to non-engineered HEK293 following systemic administration. This route was utilized as to provide the EVs access to all of the skeletal muscles, as opposed to a more localized approach such as intramuscular injection. The MYMK-EVs showed promising myotropic properties when incubated with C2C12 myotubes in vitro, with a significant alteration in the biodistribution of both EV formulations when comparing that of the mdx mouse model with the wildtype C57 mouse model in vivo. Specifically, both EV formulations showed a significant decrease in signal in the spleen and non-significant increases in the peripheral tissues, except for the brain. This suggests that circulating immune cells could play a role in the distribution of intravenous drug products and this varies with disease state. Further experiments, such as directly labeling PBMCs or administering labeled-EVs to PBMCs ex vivo and then administering the cells to mice, could validate this mechanism. Another route may be to utilize immune cells capable of transcytosis, such as leukocytes, as the EV producer line which could then be engineered to express the myotropic EV candidates. Characterization data reporting the density of the MYMK protein at the surface of the EVs would provide further context to these findings as ligand density may reasonably affect the ability of the EVs to bind to the recipient muscle cells.

Applicant observed a marked decrease in EV-derived signal in the spleen of mdx mice relative to C57 mice along with a non-significant increase in all of the peripheral tissues. This suggests a role of circulating immune cells in the redistribution of intravenously administered drug products that varies with disease state.

### Materials/Methods

### Animals

For the in vivo biodistribution experiments, 6-11 week old male C57/BL6 and mdx mice were used. For both sets of biodistribution experiments utilizing either a lipophilic or amine-reactive probe, C57 and mdx mice were randomly assigned to 3 experimental groups each: no injection control, HEK-EV injection, or MYMK-EV injection. Thus, a total of 36 mice were utilized for these experiments. Male mice were used exclusively as DMD is an X chromosome-linked genetic condition, thus it affects males at a rate of 1:3, 500-6,000 live births and largely does not affect females (1:50,000,000 live births). Mice were group housed on a 12-hour light cycle and had free access to food and water throughout the duration of the experiments. All mouse experiments conducted were approved by the University of Delaware IACUC under AUP 1387.

### Transient Transfections

For experiments involving transient transfections, Human Embryonic Kidney 293 (HEK293) cells were transfected with plasmids encoding GFP-tagged MYMK, MYMX, or M-Cad downstream of a cytomegalovirus promoter using the JetOPTIMUS cationic lipid reagent at a 1:1 ratio for 4 h per the manufacturer's instructions. 24 h later, EV purification protocol was performed. Transfection efficiency was confirmed using the 488 nm channel on the ImageExpress Pico (Molecular Devices, San Jose, CA) to obtain representative images of the endogenous GFP tag-derived signal for each transfection group.

### Generation of Stable Cell Lines for MYMK-EV Production

To generate a (HEK293) cell line that stably over-expressed MYMK, HEK293 cells were transduced with lentiviral particles (LVP) containing the MYMK transgene cloned into a pLenti-C-mGFP-P2A-Puro plasmid at a multiplicity of infection (MOI) of 10 LVP/cell. This was based on preliminary experiments that found this MOI to be sufficient. The cells were then expanded back to 90-95% confluency before subsequent passaging. The FACSAria Fusion (BD Biosciences, Woburn, MA) was used to select for stable clones displaying the highest MYMK expression as determined by the endogenous mGFP tag. The cells were subsequently expanded to 875 cm² flasks (Falcon, Corning, NY) in growth medium (GM) containing high glucose DMEM, 5% FBS and 100 U/mL of p/s. Once in the 875 cm² flasks, the cells were expanded to 90-95% and the serum-containing GM was switched to serum-free OptiMEM for EV production. Two washes with 1×PBS+10% OptiMEM or DMEM were used to remove residual serum-containing GM. The cells were incubated in the OptiMEM for 48 h before the EV-conditioned media was collected. The cells were subsequently passaged using 0.25% trypsin-EDTA (ThermoFisher, Waltham, MA) followed by inoculation into new 875 cm² flasks. Total viable cell density was routinely measured and only cells with a viability of >90% were used for EV production. Additionally, aliquots of 1.0e7 cells were collected at passages 19, 21 and 23 and stored in the liquid nitrogen vapor phase. To measure MYMK expression in the cells over the time of EV production, the aliquots were then thawed and spun at 500×g for 5 min. The pellets were resuspended in 1 ml of 1×RIPA buffer with 1× protease and phosphatase inhibitor cocktail and vortexed at max speed for 3×10 sec. 100 μl of cell lysate was loaded into a 96-well plate in triplicate and analyzed using the 488 nm channel on a fluorescent plate reader.

### EV Purification

For the HEK, MYMK, MYMX and M-Cad EVs produced following transient transfection for preliminary in vitro uptake experiments in C2C12, EVs were concentrated using the polyethylene glycol-based reagent, ExoQuick TC (Systems Biosciences, Palo Alto, CA), added at a 1:5 ratio with the conditioned media. The media-ExoQuick solutions were incubated at 4° C. overnight and then spun at 1,500×g for 30 min the following day. The EV-containing pellets were re-suspended in 100 ul of 1×PBS prior to downstream analysis.

For the HEK and MYMK-EVs produced for the biodistribution experiments, the harvested conditioned media was centrifuged at 500×g for 10 min to remove dead cells and then at 2,000×g for 20 min to remove larger cell debris. The EVs were then purified from the conditioned media via ultracentrifugation (UC) at 100,000-150,000×g for 60-90 min. In experiments in which the EVs were stained, the pellet was washed with 25 ml of 1×PBS following staining and the EVs were re-pelleted via UC following staining at 150,000×g for 60 min. All centrifugation steps were performed at 4° C.

### Nanoparticle Tracking Analysis (NTA)

A Nanosight NS300 (Malvern Panalytical, Malvern, PA), with a 532 nm green laser and NS300 FCTP Gasket (Malvern Panalytical, Malvern, PA), was used for NTA characterization of the number and size of the EVs in each sample. The EV samples were then analyzed using a camera level of 12 and a detection threshold of 10. Three 30 second videos were obtained for each sample. The EV samples were diluted to a working concentration of ˜1e9/mL for analysis and were administered into the gasket using a sterile BD Plastipak syringe (Becton Dickinson S.A., Madrid, Spain).

### Flow Cytometry

A FACS Aria Fusion was used to examine the efficiency with which each native myotropic protein candidate was incorporated into EV. To do so each EV sample was labeled using ExoGlow Red (Systems Biosciences, Palo Alto, CA) following the manufacturer's instructions. Briefly, dye stock was added to each sample at a 1:500 dilution and incubated at 37° C. for 20 min while shaking at 350 rpm. ExoQuick TC was then add to each sample at a 1:5 ratio and incubated at 4° C. overnight. The next day, the samples were re-pelleted at 1,500×g for 30 min at 4° C. The supernatant was then carefully aspirated and the pellets were re-suspended in 100 μl of 1×PBS. For flow cytometry, the samples were further diluted 1:100 in 0.1 m-filtered 1×PBS and analyzed using a FACS Aria Fusion. Specifically, the GFP and PE-A laser lines were utilized to measure the signal from the GFP-tagged recombinant proteins (MYMK, MYMX, M-Cad) and the ExoGlow Red stain, respectively. Gating was established by first running unstained, non-engineered HEK293 EVs and modifying the gating parameters to exclude this signal. The labeled samples from each of the cell lines were then run through the instrument until 30,000 total events were captured. The data were analyzed using FCS Express to measure the percentage of events that were positive for both GFP and the ExoGlow Red stain to obtain an estimate of the percentage of EVs that were positive for the recombinant proteins following transient transfection.

### Screening of Native Myotropic EV Candidates

For the preliminary screening of the native myotropic EV candidates, EVs were labeled with 40 M carboxyfluorescein diacetate succinimidyl ester (CFDA-SE) for 2 hours at 37° C. To remove unbound dye, the EV samples were then incubated with ExoQuick-TC at a 1:5 sample:reagent ratio at 4° C. overnight. The samples were then centrifuged at 1,500×g for 30 min to re-pellet the labeled EVs. The labeled pellet was re-suspended in 100 μl 1×PBS prior to downstream experiments. C2C12 myoblasts were seeded in half surface area 96-well plates (Greiner Bio-One, Monroe, NC) at 10,000 cells/well in growth medium containing 10% FBS, 1% p/s in DMEM. After 24 hours, the medium was switched to differentiation medium containing 2% horse serum rather than FBS. After six days in the DM, the C2C12 myoblasts had fully differentiated into myotubes at which time they were incubated with 5.0e8 of each EV formulation as determined by NTA. After 24 hours, the medium was changed to DM containing Live-Cell NucBlue for 20 min at 37° C. to label the nuclei. The C2C12 myotubes were then imaged on an ImageExpress Pico and the images were analyzed to measure the CFSE-derived signal in the 488 nm channel relative to each nucleus.

### BCA Protein Assay

The BCA protein assay was used to determine the protein concentration of EV samples following the manufacturer's instructions. EVs were lysed using 1×RIPA buffer and 1× protease and phosphatase inhibitor cocktail. Protein concentrations were determined using a BioTek Synergy 2 plate reader (Agilent, Santa Clara, CA). The derived protein concentration was compared to the particle count obtained from NTA to determine the protein/particle ratio as a measure of sample purity.

### Western Blotting

SDS-PAGE was performed with a Quadra Mini Vertical Blotting System (CBS Scientific, San Diego, CA). Samples were mixed with 4× lithium dodecyl sulfate (LDS) sample buffer along with 1.0% Triton X-100, and 15 mM dithiothreitol. The samples were then heated at 95° C. for 10 min. Once denatured, the samples were then loaded into a RunBlue 4-12% TEO-Tricine Protein Gel and run at 130 V in RunBlue TEO-Tricine run buffer for 1.5 hr with a stir bar and ice pack. A Power Blotter system (Thermo Fisher Scientific, Waltham, MA) was used to transfer the proteins on the gel to a nitrocellulose membrane with 0.2 μm pore size for 10 min. The membranes were blocked using Omniblok™ overnight at 4° C. with gentle rocking. After blocking, the membranes were incubated overnight at 4° C. with primary antibody solution (1:1000 dilution for anti-Alix and anti-Tsg101, 1:2000 dilution for anti-GAPDH, and 1:5000 dilution for β-actin) with gentle rocking. After primary antibody incubation, the membranes were washed three times with tris buffered saline (TBS) containing 0.1% Tween-20 (TBS-T) for 5 min per wash. The membranes were then incubated with HRP-conjugated secondary antibodies (anti-mouse or anti-rabbit depending on primary antibody species) for 1 hr at RT (1:100000) with gentle rocking. Membranes were washed two times with TBS-T for 5 min per wash and two times with TBS for 5 min again. The blots were incubated with enhanced chemiluminescent (ECL) substrate to develop and then imaged on and Odyssey infrared imaging system for analysis.

### Fluorescent Plate Reader Assays

Cell and EV lysates were generated as described previously. Equal amounts of cell and EV lysates were loaded into a 96-well plate and analyzed on the BioTek Synergy 2 plate reader using the 488 nm channel to measure the MYMK expression in both the cell and EV lysates throughout EV production.

### Transmission Electron Microscopy (TEM)

The EV samples were concentrated to ˜1.0e11-2.0e11 particles/mL. 400 mesh carbon-coated copper grids were floated onto each sample. The grids were washed with four drops of water and negatively stained with 2% uranyl acetate (aq). Prior to the addition of the sample, the grids were glow discharged with a Pelco easiGlow glow discharge unit (Ted Pella Inc, Redding, CA), making the carbon film hydrophilic. The samples were then imaged using a Zeiss Libra 120 transmission electron microscope (Oberkochen, Germany) operating at 120 kV. Images were obtained with a Gatan Ultrascan 1000 CCD camera (Warrendale, PA).

### In Vivo Biodistribution of MYMK and HEK293 EVs

Biodistribution of MYMK and non-engineered HEK293 EVs were assessed using two sets of experiments. In the first set of experiments, EVs were labeled with 5 μM DiD for 20 min at 37° C. A preliminary experiment revealed that free DiD not bound to EVs floats to the surface of PBS under UC at 150,000×g for 1 h, thus it was determined UC was a sufficient method to remove unbound dye from the sample and pellet the DiD-labeled EVs. Following labeling, NTA was utilized to measure the concentration of the EVs. 1.2e11 labeled EVs were administered to both C57/BL6 and mdx mice (N=3 male mice/group, 6-11 weeks old) via tail vein injection. After 24 hours, the mice were anesthetized using 3% isoflurane gas. Once under anesthesia, the gastrocnemius, heart, diaphragm, brain, liver, spleen and kidneys were harvested from the mice and washed with 1×PBS. The organs were then kept in 1×PBS on ice as the others were harvested. Immediately following the dissection, the organs were imaged on an IVIS Lumina III (Perkin Elmer, Waltham, MA) using excitation 689 nm and emission 713 nm. Additionally, the whole organs or sections of the larger organs were homogenized in 1 ml of TPER or NPER (brain) using 1.4 mm ceramic beads (Omni International, Kennesaw, GA) in a Mini Bead Mill (VWR, Radnor, PA) and 100 μl of tissue homogenate was loaded into a 96-well plate and analyzed using a fluorescent plate reader to obtain quantitative fluorescence data, normalized to tissue weight (g).

### Statistical Analysis

For initial in vitro experiments, a one-way ANOVA with a Tukey post hoc test for multiple comparisons was utilized to test for statistical significance. For the in vivo biodistribution experiments, a two-way ANOVA with a Tukey post hoc multiple comparisons was utilized to test for statistical significance. For all in vitro experiments, three independent experiments were conducted. The data are presented as the average of the means+/−standard error (SE). For animal experiments, 3 mice were utilized per experimental group, equating to a total of 36 mice. Data for each experimental group are presented as the average of the means+/−SE. A p value<0.05 was considered significant for all experiments. All statistical analyses were conducted in GraphPad Prism 9.4.1.

### Example 2. Chimeric Myotropic Extracellular Vesicles

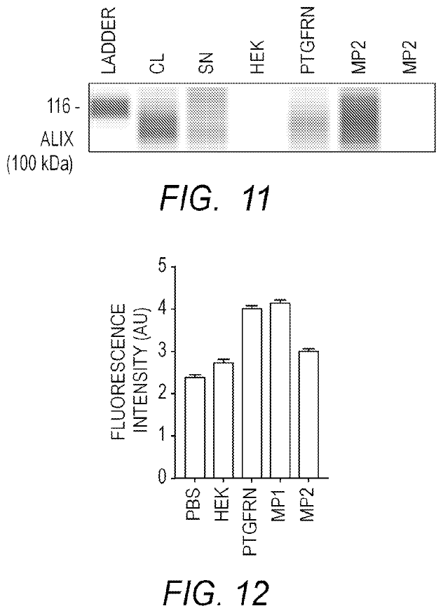

Applicant examined the myotropic properties of chimeric myotropic EV candidates in vitro by displaying two different myotropic peptides on the EV membrane protein prostaglandin F2 receptor inhibitor (PTGFRN) and incubating the EVs with muscle and non-muscle cell types. Plasmids encoding either MP-1 or MP-2 fused to the extracellular domain of PTGFRN (Reference Sequence NM_020440.4, NP_065173.2) were transfected into HEK293 cells followed by antibiotic selection and FACS. (FIG. 11) EVs were labeled with an amine-reactive dye and incubated with muscle and non-muscle cell-types in vitro.

Surprisingly, the EVs expressing the PTGFRN protein alone demonstrated the highest degree of myotropism relative to non-engineered EVs and those expressing the myotropic peptides. Analysis of the PTGFRN amino acid sequence revealed that it has predicted functions in myoblast fusion and in light of the current data this functionality may impart myotropic properties to this protein. In addition, Applicant did see roughly fourfold greater protein/particle numbers(?) in the HEK and MP2-EV groups as compared to the PTGFRN and MP1-EV groups, likely due to the increased EV production noted in the latter two groups. This is unlikely to have affected the results, as the dose of EVs administered to each cell-type was normalized to the quantity of fluorescently labeled protein in each sample. Biodistribution studies could validate potential myotropic properties of these EVs.

In this example, EVs were stained with the amine-reactive dye CellTracker Red (CT-R) CMTPX (FIG. 12). The signal from these EVs could not be reliably detected above the background autofluorescence in tissue. To ensure the labeling method worked as expected, the EVs were incubated with HEK293 cells in vitro and uptake of the fluorescent cargo was confirmed. Thus, these data suggest possible limitations when using an amine-reactive probe and tracking EV biodistribution by examining the labeled protein delivered to each tissue.

### Methods

In this example, experiments examined the intracellular delivery of EV-derived protein. Unless otherwise specified, materials and methods are as described in Example 1. EV luminal proteins were stained with 5 μM CT-R for 30 min at 37° C. The unbound dye was removed via elution through PD-10 desalting columns following the manufacturer's instructions (Cytiva Life Sciences, Marlborough, MA). The stained EVs were then re-pelleted at 150,000×g for 1 h and resuspended in 1×PBS. In the first set of biodistribution experiments, NTA-derived particle count was used to determine the EV dose. In the second set of experiments, a fluorescent plate reader was used to normalize the doses of EVs due to a discrepancy noted in Example 1 between the NTA particle count and the fluorescent signal. Thus, the EVs were produced using the equivalent number of cells as in the first set of experiments of Example 1 and then the fluorescence intensity was used to determine the volume of sample injected. After 24 hours the same organs were harvested as in the first set of experiments; however, the organs were frozen in OCT freezing compound (Electron Microscopy Sciences, Hatfield, PA) using a copper block placed in liquid nitrogen. The samples were then cryo-sectioned prior to immunohistochemistry (HC). The sections were fixed in 4% PFA followed by three 5 min washes with 1×PBS prior to blocking with 1% BSA for 30 min at RT. 25 μl of Phalloidin-488 was then added to 1% BSA in 1×PBS and 50 μl was add to each tissue section for 30 min at RT. This was removed with three 5 min washes with 1×PBS. After the final wash, all of the PBS was removed and one drop of Prolong Diamond Antifade Mountant with DAPI was added to each section prior to placement of the coverslip. The slides were allowed to cure overnight prior to imaging, per the instructions for the mountant. The slides were imaged on an Image Express Pico using a 20× objective lens and the following excitation/emission wavelengths were used: 577/602 nm for CT-R, 484/501 nm channel for Phalloidin-488, and 358/461 nm for DAPI. For the in vitro experiment to verify ability of CT-R-labeled EVs to deliver labeled-protein into cells, HEK293 cells were seeded at 10,000 cells/well in a half surface area 96-well plate. 4 hours later the growth medium was changed to growth medium containing CT-R-labeled EVs determined using a fluorescent plate reader. 24 h later the cells were stained with 5 M DiO for 15 min at 37° C. followed by Live-Cell NucBlue at 37° C. Following staining, the cells were imaged on the ImageExpress Pico using the 358/461 nm for the Live Cell NucBlue, 484/501 nm for DiO, and 577/602 nm for CT-R. Additionally, dystrophin protein expression was stained for in the gastrocnemius muscle. Tissue sections were fixed using 4% PFA for 10 minutes at RT. This was washed off by 3×5 min washes with 1×PBS. The sections were then blocked using 1% BSA for 30 min at RT. This was dumped off without washing followed by incubation with Rabbit polyclonal Anti Dystrophin C-Terminus primary antibody at a 1:100 dilution in 1% BSA overnight at 4 C. The next day, the primary antibody solution was washed with 3×5 min washes with 1×PBS. This was followed by incubation with Goat anti-rabbit AlexaFluor 647 IgG F(ab′)2 frag at a 1:200 dilution in 1% BSA at RT for 1 hour. This was then washed off with 3×5 min washes in 1×PBS and mounted using Prolong Diamond Antifade with DAPI. The slides were allowed to cure overnight at RT prior to being imaged on the Image Express Pico the following day. The Cy5 channel was utilized to image dystrophin and the DAPI channel was utilized to image the nuclei.

### CONCLUSION

Overall, the MYMK-EV formulation showed promise in its ability to enhance delivery of protein into C2C12 myotubes in vitro but did not significantly alter the biodistribution toward skeletal or cardiac muscle in vivo. Unexpectedly, a significant difference in the biodistribution of EVs administered in mdx mice relative to C57 mice was observed. Most notably, there was a significant decline in signal in the spleen of the mdx mice relative to the C57 mice which occurred simultaneously with a redistribution of the signal to the peripheral tissues of the mdx mouse. It may be that the immune system and the inflammatory state of the tissues is involved in this redistribution given that the spleen is a reservoir for various immune cells which may feasibly uptake EVs in the circulation and migrate to tissues in a chronic state of inflammation. Lack of consistency of measurement of a signal above background from the tissues harvested following administration of the CT-Red EVs may have been due to a number of factors involving the low quantity of protein in each EV, the potential for the EV-derived protein to be degraded following administration and the short EV half-life relative to the commonly used 24 h incubation period following administration of the EVs. Finally, when comparing the particle concentration between the DiD-EVs and CT-Red EVs, the DiD-EVs exhibited a drastically elevated particle concentration. This is likely due to some sort of artifact and should be taken into account when choosing doses of EVs to administer in vivo. Choosing an EV dose based on fluorescence intensity may be a more accurate and precise method relative to particle count. Overall, these examples provide a potentially interesting mechanism of how EV cargoes are distributed following intravenous delivery in a diseased state as compared to a non-diseased state, with circulating immune cells potentially playing a critical role. In addition, the experiments using an amine-reactive dye to track the biodistribution of EVs provide a number of practical insights into staining and dosing EV samples for in vivo biodistribution studies.

The foregoing examples are illustrative of the present invention and are not to be construed as limiting thereof. Although the invention has been described in detail with reference to preferred embodiments, variations and modifications exist within the scope and spirit of the invention as described and defined in the following claims.

1. A composition comprising extracellular vesicles isolated from cell culture medium of cultured cells modified to express a muscle targeting membrane protein, or fragment thereof, wherein the extracellular vesicles are targeted to a muscle cell or tissue.

2. The composition of claim 2, wherein the muscle targeting membrane protein, or fragment thereof, is a protein listed in Table 1.

3. The composition of claim 2, wherein the muscle targeting membrane protein, or fragment thereof, is MyoMaker (MYMK), MyoMixer (MYMX), M-Cadherin (M-CAD), or a combination thereof.

4. The composition of claim 2, wherein the muscle targeting membrane protein, or fragment thereof, is prostaglandin F2 receptor inhibitor (PTGFRN).

5. The composition of claim 4, wherein the PTGFRN is fused to a synthetic myotropic peptide.

6. The composition of claim 5, wherein the synthetic myotropic peptide is ASSLNIA (MP1)(SEQ ID NO:1) or RRQPPRSISSHP (MP2)(SEQ ID NO:2).

7. The composition of claim 1, wherein the extracellular vesicles further comprise one or more therapeutic agents.

8. The composition of claim 7, wherein the cells have been modified to contain a higher level of the one or more therapeutic agents than unmodified cells.

9. The composition of claim 7, wherein the one or more therapeutic agents is a protein, RNA (mRNA, siRNA, microRNA, lncRNA), DNA, plasmid, viral vector (e.g., AAV), exon skipping compound, or any combination thereof.

10. The composition of claim 7, wherein the one or more therapeutic agents is selected from the agents listed in Table 2.

11. The composition of claim 7, wherein the one or more therapeutic agents is endogenously expressed in the cultured cells.

12. The composition of claim 7, wherein the one or more therapeutic agents or a nucleic acid encoding the one or more therapeutic agents have been introduced into the cells.

13. (canceled)

14. The composition of claim 1, wherein the cells are HEK293 cells.

15. A method of treating a muscular disorder, condition, or damage in a subject in need thereof, comprising:
administering a therapeutically effective amount of the composition of claim 1 to the subject;
thereby treating the muscular disorder, condition, or damage.

16-18. (canceled)

19. A method of making the composition of claim 1, comprising
introducing a nucleic acid encoding the targeting membrane protein, or fragment thereof, into the cell;
culturing the cells in cell culture medium to thereby express the target membrane protein; and
isolating the composition comprising the extracellular vesicles from the cell culture medium.

20. (canceled)

21. A method of targeting one or more therapeutic agents to muscle cells, comprising contacting the muscle cells with extracellular vesicles isolated from cell culture medium of cultured cells modified to express a muscle targeting membrane protein, or fragment thereof, and one or more therapeutic agents.

22-26. (canceled)

27. The method of claim 21, wherein the muscle targeting membrane protein, or fragment thereof, is MyoMaker (MYMK), MyoMixer (MYMX), or M-Cadherin (M-CAD), or a combination thereof.

28. The method of claim 21, wherein the targeting membrane protein, or fragment thereof, comprises prostaglandin F2 receptor inhibitor (PTGFRN).

29. The method of claim 28, wherein the extracellular domain of PTGFRN is fused to a synthetic myotropic peptide.

30. The method of claim 29, wherein the synthetic myotropic peptide is ASSLNIA (MP1)(SEQ ID NO:1) or RRQPPRSISSHP (MP2) (SEQ ID NO:2).

31. (canceled)


SEQUENCE LISTING (2 sequences)

SEQ ID NO: 1 - protein, 7 aa, synthetic construct
ASSLNIA

SEQ ID NO: 2 - protein, 12 aa, synthetic construct
RRQPPRSISSHP